(19) United States
(12) Patent Application Publication
(10) Pub. No.: US 2025/0025048 A1
(43) Pub. Date: Jan. 23, 2025

(54) Liver Viscoelastic Changes and Biomarkers for Cancer Invasion

(71) Applicant: The Board of Trustees of the Leland Stanford Junior University, Stanford, CA (US)

(72) Inventors: Natalie J. Torok, Redwood City, CA (US); Ovijit Chaudhuri, Redwood City, CA (US); Weiguo Fan, Redwood City, CA (US)

(21) Appl. No.: 18/714,022

(22) PCT Filed: Dec. 2, 2022
(86) PCT No.: PCT/US2022/080849

§ 371 Date: May 28, 2024

Related U.S. Application Data
(60) Provisional application No. 63/285,623, filed on Dec. 3, 2021.

(51) Int. Cl. A61B 5/00; A61B 8/08

(52) U.S. Cl. CPC A61B 5/0036; A61B 8/485; A61B 2503/40

(57) ABSTRACT

Methods of prognosis, diagnosis, therapy selection, drug screening, and monitoring treatment of hepatocellular carcinoma (HCC) are provided. Increased viscoelasticity of liver tissue is associated with HCC progression; therefore, measurements of viscoelasticity of liver tissue can be used to provide an indication of the risk of HCC progression in a patient. In addition, animal models of hydrodynamically induced HCC and methods of using such animal models for research and development of therapeutics for treating HCC are also provided.

### CROSS REFERENCE TO RELATED APPLICATIONS

This application claims benefit of U.S. Provisional Patent Application No. 63/285,623, filed Dec. 3, 2021, which application is incorporated herein by reference in its entirety.

### INCORPORATION BY REFERENCE OF A SEQUENCE LISTING

A Sequence Listing is provided herewith as a Sequence Listing XML file, “STAN-1917WO”, created on Nov. 22, 2022, and having a size of 2,360 bytes. The contents of the Sequence Listing XML file are incorporated by reference herein in their entirety.

### BACKGROUND OF THE INVENTION

Non-alcoholic steatohepatitis (NASH) is the most common liver disease, leading to cirrhosis and hepatocellular carcinoma (HCC). HCC is one of the most common cancers and has a dismal prognosis as currently available medical treatment only improves survival by a few months. The current guidelines only focus on screening patients for HCC with cirrhotic stage liver disease. It is recognized, however, that 30-40% of all NASH-related HCCs occur in non-cirrhotic livers. These patients often present at a late stage when they are not eligible for liver transplant, and other treatment modalities are very limited. Therefore, there is an urgent need to develop new screening strategies and develop new biomarkers to improve survival of these patients.

### SUMMARY OF THE INVENTION

Methods of prognosis, diagnosis, therapy selection, drug screening, and monitoring treatment of HCC are provided. Increased viscoelasticity of liver tissue is associated with HCC progression; therefore, measurements of viscoelasticity of liver tissue can be used to provide an indication of the risk of HCC progression in a patient. In addition, animal models of hydrodynamically induced HCC and methods of using such animal models for research and development of therapeutics for treating HCC are also provided.

In one aspect, a method of diagnosing HCC in a patient is provided, the method comprising measuring viscoelasticity of liver tissue from the patient, wherein increased viscoelasticity of the liver tissue of the patient compared to reference value ranges for viscoelasticity of liver tissue from a control subject indicates that the patient is at risk of developing HCC or has a positive diagnosis for HCC.

In certain embodiments, tissue viscoelasticity is measured using atomic force microscopy, magnetic resonance elastography, or ultrasound.

In certain embodiments, the method further comprises treating the patient for the HCC, if the patient has a positive diagnosis for the HCC based on the measured viscoelasticity. In some embodiments, the method further comprises performing medical imaging of the liver to confirm the positive diagnosis for HCC prior to said treating the patient. Exemplary medical imaging techniques include, without limitation, magnetic resonance imaging (MRI), positron emission tomography (PET), single photon emission computed tomography (SPECT), computed tomography (CT), ultrasound imaging (UI), optical imaging (OI), photoacoustic imaging (PI), fluoroscopy, and fluorescence imaging.

In another aspect, a method of generating a non-human animal model of non-alcoholic steatohepatitis with hydrodynamically induced HCC is provided, the method comprising: administering an advanced glycation end-product (AGE) to the non-human animal, wherein liver tissue viscoelasticity increases in the non-human animal.

In certain embodiments, the non-human animal is a mammal. In some embodiments, the mammal is a rodent (e.g., mouse, rat, or guinea pig) or a non-human primate (e.g., chimpanzee, macaque, monkey, or ape).

In another aspect, a non-human animal model of non-alcoholic steatohepatitis with hydrodynamically induced HCC, produced by a method described herein, is provided.

In another aspect, a method of screening a candidate agent is provided, the method comprising: administering the candidate agent to the non-human animal model of non-alcoholic steatohepatitis with hydrodynamically induced HCC, described herein; and measuring viscoelasticity of liver tissue in the non-human animal model of non-alcoholic steatohepatitis with hydrodynamically induced HCC.

In certain embodiments, the method further comprises testing if the candidate agent is an AGE inhibitor or a crosslink breaker or increases expression or activity of AGER1.

In certain embodiments, the method further comprises testing if the candidate agent has anti-cancer activity.

In another aspect, an isolated cancerous liver cell from the non-human animal model of non-alcoholic steatohepatitis with hydrodynamically induced HCC is provided.

In another aspect, a method of screening a candidate agent is provided, the method comprising: contacting the cancerous liver cell from the non-human animal model of non-alcoholic steatohepatitis HCC with the candidate agent; and measuring the viscoelasticity of the ECM of the cancerous liver cell.

In certain embodiments, the method further comprises testing if the candidate agent is an AGE inhibitor or a crosslink breaker or increases expression or activity of AGER1.

In certain embodiments, the method further comprises testing if the candidate agent has anti-cancer activity.

In another aspect, a method of treating a subject for HCC is provided, the method comprising administering a therapeutically effective amount of an AGE inhibitor (e.g., pyridoxamine), a crosslink breaker that cleaves crosslinks formed by an AGE (e.g., alagebrium (3-phenacyl-4,5-dimethylthiazolium chloride, ALT-711)), or a combination thereof to the subject.

In another aspect, a method of treating a subject for HCC is provided, the method comprising administering a vector comprising a promoter operably linked to a nucleotide sequence comprising a coding sequence encoding AGER1 to the subject, wherein a therapeutically effective amount of the AGER1 is expressed in vivo in the subject.

In certain embodiments, the vector is administered intravenously, intra-arterially, or locally into the liver.

In certain embodiments, the vector is a viral vector. In some embodiments, the viral vector is an adeno-associated virus (AAV) vector.

In certain embodiments, the promoter is a constitutive or inducible promoter.

In another aspect, a method of providing a subject with AGER1 is provided, the method comprising introducing a vector comprising a promoter operably linked to a coding sequence encoding the AGER1 into a liver cell, wherein the cell expresses the AGER1 in vivo in the subject in an effective amount sufficient to have anti-cancer activity.

In certain embodiments, the vector is introduced into the cell ex vivo or in vivo.

In certain embodiments, the vector is a viral vector. In some embodiments, the viral vector is an adeno-associated virus (AAV) vector.

In certain embodiments, the promoter is a constitutive or inducible promoter.

In another aspect, a composition for use in a method of treating HCC is provided, the composition comprising an advanced glycation end product (AGE) inhibitor (e.g., pyridoxamine), a crosslink breaker that cleaves crosslinks formed by an AGE (e.g., alagebrium), or a combination thereof. In some embodiments, the composition further comprises a pharmaceutically acceptable excipient.

In another aspect, a composition for use in a method of treating HCC is provided, the composition comprising AGER1 or a vector comprising a promoter operably linked to a coding sequence encoding AGER1. In some embodiments, the composition further comprises a pharmaceutically acceptable excipient.

### BRIEF DESCRIPTION OF THE DRAWINGS

FIGS. 1A-1B. ECM viscoelasticity increases in an advanced glycation end product (AGE) and hepatocyte RAGE-dependent manner. Schematic presentation of the methods used to assess mechanical responses to indentation by an atomic force microscope (AFM) probe. Force-distance curve is generated when the probe indents the sample until a defined force is reached (blue, approach curve) and the cantilever is retracted (red, retraction curve). The hysteresis between the approach and retraction curves indicates sample viscosity (FIG. 1A). Force curves using livers from HiAD-fed mice exhibit larger hysteresis area (arrow, the area between approach and retraction force curves) compared to chow or FFD-fed mice. The hysteresis is also reduced in PM-treated (60 mg/kg daily, i.p.) or in RAGE HepKO mice (FIG. 1B, N=2-3/group).

FIGS. 2A-2D. Atomic force microscopy on non-cirrhotic liver samples from NASH patients show higher viscoelasticity in patients with type 2 diabetes mellitus (T2DM). Force curves show larger hysteresis area (the area between approach and retraction force curves) in patients with T2DM (FIG. 2A). Trapz function in MATLAB was used to measure the hysteresis area in (FIG. 2A), N=10 areas in each group, each sample (FIG. 2B, mean±SEM, p=0.03, ANOVA, Tukey's post hoc). Representative stress relaxation response curves show faster stress relaxation in NASH patients with T2DM (FIG. 2C). In this test, a constant strain (3 nN) is applied and the stress in response to strain is measured for 10 s. Liver AGEs increased in patients with T2DM (n=3 in each group (FIG. 2D). Mean±SEM, p<0.01, t-test).

FIGS. 3A-3D. Using rheometry, mice on HiAD exhibited faster relaxation times (high viscoelasticity) that was reversed by PM, and alagebrium (ALT-711). Stiffness did not change. The plate was lowered to touch the sample and 100˜300 Pa of initial force was applied. Dynamic time sweep test (2% constant strain, oscillation frequency 1 radian/s, measurements taken for 600 s) was done first to collect the shear storage modulus (stiffness, G′) and loss modulus (G″) (FIG. 3A). The storage modulus (stiffness) did not show significant change (FIG. 3B). Stress relaxation test was performed for 600 s with an initial 10% strain. Livers from HiAD-fed mice exhibited faster stress relaxation compared to chow, FFD-fed mice or those treated with the AGE inhibitor, PM, or ALT-711 (crosslink breaker). Stress was normalized by the initial stress (FIG. 3C). Quantification of timescale at which the stress is relaxed to half its original value (τ_{1/2}), from the tests in FIG. 3C (FIG. 3D). (N=2-4, Data are represented as mean±SEM; one way ANOVA followed by post-hoc Tukey test *p<0.05, **p<0.01, ***p<0.001).

FIGS. 4A-4F. RAGE^{HepKO} and AGER1 reconstituted mice exhibit lower number of GS/c-Myc positive transformed cells. RAGE fl/fl or RAGE^{HepKO} mice were fed HiAD for 7 weeks, HD-injected, and sacrificed 4 weeks later (FIG. 4A, bar=100 μm). AGER1 was reconstituted by injecting AAV8-TBG-AGER1 mice via their tail vein 2w prior to the HD injection, as control AAV8-TBG-GFP were used (FIG. 4B). GS/c-Myc positive cells decreased in RAGE^{HepKO} mice and AAV8-AGER1 mice (FIGS. 4A, 4B). RT-qPCR shows increase in AGER1 levels (FIG. 4C). Rheometry data demonstrate that the storage modulus (stiffness) did not change (FIG. 4D). Livers from RAGE^{HepKO} and AAV8-TBG-AGER1 mice exhibited faster stress relaxation compared to control mice. Stress was normalized by the initial stress (FIG. 4E). Quantification of timescale at which the stress is relaxed to half its original value (τ½), from stress relaxation tests in FIG. 4C. (FIG. 4F). (N=4-6, Data are represented as mean±SEM; one way ANOVA followed by post-hoc Tukey test *p<0.05, **p<0.01, ***p<0.001).

### DETAILED DESCRIPTION OF THE INVENTION

Methods of prognosis, diagnosis, therapy selection, drug screening, and monitoring treatment of hepatocellular carcinoma (HCC) are provided. Increased viscoelasticity of liver tissue is associated with HCC progression; therefore, measurements of viscoelasticity of liver tissue can be used to provide an indication of the risk of HCC progression in a patient. In addition, animal models of hydrodynamically induced HCC and methods of using such animal models for research and development of therapeutics for treating HCC are also provided.

Before the present compositions, methods, and animal models are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “an agent” includes a plurality of such agents and reference to “the vector” includes reference to one or more vectors and equivalents thereof, e.g., viral vectors, plasmids, and constructs, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “animal” is used herein to include all vertebrate animals, except humans. The term also includes animals at all stages of development, including embryonic, fetal, neonate, and adult stages. Animals may include any member of the subphylum Chordata, including, without limitation, non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like.

As used herein, the term “determining” refers to both quantitative and qualitative determinations and as such, the term “determining” is used interchangeably herein with “assaying,” “measuring,” and the like.

The terms “advanced glycosylation end products”, “advanced glycation end products”, and “AGEs” are used interchangeably and refer to glycated proteins, nucleic acids, or lipids. AGEs are formed non-enzymatically by condensation between carbonyl groups of reducing sugars (e.g., reducing aldoses and reducing ketoses) and free amine groups of proteins, nucleic acids, or lipids to produce early glycosylation end products, which subsequently undergo further chemical rearrangement to produce stable advanced glycosylation end-products. AGEs include, but are not limited to, glucose-derived AGEs (Glu-AGEs), fructose-derived AGEs (Fru-AGEs), glyoxal-derived AGEs (GO-AGEs), methylglyoxal-derived AGEs (MGO-AGEs), glyceraldehyde-derived AGEs (Glycer-AGEs), glycolaldehyde-derived AGEs (Glycol-AGEs), and 3-deoxyglucosone-derived AGEs (3-DG-AGEs). AGEs may be generated through various pathways, including, without limitation, the Hodge pathway, Namiki pathway, or Wolff pathway. See, e.g., Twarda-Clapa et al. (2022) Cells 11(8): 1312, Singh et al. (2001) Diabetologia 44(2): 129-146, Perrone et al. (2020) Oxid. Med. Cell Longev. 2020:3818196; herein incorporated by reference.

The term “AGE inhibitor” as used herein refers to any molecule (e.g., small molecule inhibitor, protein, polypeptide, peptide, fusion protein, inhibitory nucleic acid (e.g., siRNA, miRNA, antisense nucleic acid), peptide nucleic acid, antibody, antibody mimetic, or aptamer)) that reduces AGE formation, reduces AGE deposition in the liver, sequesters or removes AGEs, reduces levels of AGEs in the liver, or reduces AGE-mediated toxicity. For example, an AGE inhibitor may reduce the level of an AGE in the liver by 10%, 20%, 30%, 40%, 50%, 60%, 70%, 80%, 90%, 100%, or any amount in between as compared to native or control levels.

An “effective amount” of an AGE inhibitor (e.g., pyridoxamine) is an amount sufficient to reduce formation/deposition of AGEs in the liver, reduce levels of AGEs in the liver, or reduce AGE-mediated toxicity. An effective amount can be administered in one or more administrations, applications, or dosages.

The term “crosslink breaker” as used herein refers to any molecule (e.g., small molecule or protein) that can cleave covalent AGE crosslinks between proteins, nucleic acids, or lipids. For example, a crosslink breaker may reduce the level of AGE cross-linked proteins, AGE cross-linked nucleic acids, or AGE cross-linked lipids by 10%, 20%, 30%, 40%, 50%, 60%, 70%, 80%, 90%, 100%, or any amount in between as compared to native or control levels.

An “effective amount” of a crosslink breaker (e.g., alagebrium) is an amount sufficient to reduce levels of AGE cross-linked proteins, AGE cross-linked nucleic acids, or AGE cross-linked lipids. An effective amount can be administered in one or more administrations, applications, or dosages.

The term “administering” is intended to include routes of administration which allow the agent to perform its intended function of inhibiting formation of AGEs, reducing levels of AGEs in the liver, reducing toxicity of AGEs, cleaving crosslinks between AGEs and proteins (e.g., extracellular matrix proteins), nucleic acids, or lipids, and/or inhibiting growth/proliferation of liver cancer cells. The agent can be administered by any suitable route of administration, including orally, via injection (subcutaneously, intravenously, or intramuscularly), by infusion, or locally. Additional modes of administration are also contemplated, such as intra-arterial, percutaneous, intraperitoneal, pulmonary, intralesion, transdermal, transmucosal, and so forth. When administering an agent by injection, the administration may be by continuous infusion or by single or multiple boluses. Depending on the route of administration, the agent can be coated with or disposed in a selected material to protect it from natural conditions which may detrimentally affect its ability to perform its intended function. The agent may be administered alone, or in conjunction with a pharmaceutically acceptable carrier. Further, the agent may be coadministered with a pharmaceutically acceptable carrier. The agent also may be administered as a prodrug, which is converted to its active form in vivo.

The term “affinity” refers to the strength of binding and can be expressed quantitatively as a dissociation constant (Kd). For example, in certain embodiments, an AGE inhibitor or crosslink breaker interacts preferentially with an AGE but, nonetheless, may be capable of binding other glycosylated molecules at a weak, yet detectable, level (e.g., 10% or less of the binding shown to the AGE of interest). Typically, weak binding, or background binding, is readily discernible from the preferential interaction with the AGE of interest, e.g., by use of appropriate controls.

The terms “tumor,” “cancer” and “neoplasia” are used interchangeably and refer to a cell or population of cells whose growth, proliferation or survival is greater than growth, proliferation or survival of a normal counterpart cell, e.g. a cell proliferative, hyperproliferative or differentiative disorder. Typically, the growth is uncontrolled. The term “malignancy” refers to invasion of nearby tissue. The term “metastasis” or a secondary, recurring or recurrent tumor, cancer or neoplasia refers to spread or dissemination of a tumor, cancer or neoplasia to other sites, locations or regions within the subject, in which the sites, locations or regions are distinct from the primary tumor or cancer. Neoplasia, tumors and cancers include benign, malignant, metastatic and non-metastatic types, and include any stage (I, II, III, IV or V) or grade (G1, G2, G3, etc.) of neoplasia, tumor, or cancer, or a neoplasia, tumor, cancer or metastasis that is progressing, worsening, stabilized or in remission. In particular, the terms “tumor,” “cancer” and “neoplasia” include carcinomas, such as squamous cell carcinoma, adenocarcinoma, adenosquamous carcinoma, anaplastic carcinoma, large cell carcinoma, and small cell carcinoma.

“Hepatocellular carcinoma” refers to any type of hepatocellular carcinoma of any stage (e.g., stages IA, IB, II, IIIA, IIIB, IVA, and IVB according to the American Joint Committee on Cancer, or stages 0 (very early stage), A (early stage), B (intermediate stage), C (advanced stage), and D (terminal stage) according to Barcelona Clinic Liver Cancer staging classification, or the like), grade (e.g., low grade (G1 and G2) and high grade (G3 and G4)), or subtype, including, without limitation, fibrolamellar hepatocellular carcinoma, pseudoglandular hepatocellular carcinoma (adenoid), pleomorphic (giant cell) hepatocellular carcinoma, clear cell hepatocellular carcinoma, sarcomatoid hepatocellular carcinoma, steatohepatitic hepatocellular carcinoma, macrotrabecular-massive hepatocellular carcinoma, lymphocyte-rich hepatocellular carcinoma, scirrhous hepatocellular carcinoma, cirrhosis-like hepatocellular carcinoma, combined hepatocellular carcinoma-cholangiocarcinoma, lymphoepithelioma-like hepatocellular carcinoma, and transitional liver cell tumors. The term includes nodular, massive, and diffuse types of hepatocellular carcinoma.

By “anti-tumor activity” or “anti-cancer activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Such activity can be assessed using animal models.

By “therapeutically effective dose or amount” of an AGE inhibitor and/or a crosslink breaker that cleaves crosslinks formed by an AGE, and/or a recombinant polynucleotide comprising a coding sequence encoding an AGER1 protein is intended an amount that, when administered as described herein, brings about a positive therapeutic response in treatment of hepatocellular carcinoma, such as an amount having anti-tumor activity. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

The term “tumor response” as used herein means a reduction or elimination of all measurable lesions. The criteria for tumor response are based on the WHO Reporting Criteria [WHO Offset Publication, 48-World Health Organization, Geneva, Switzerland, (1979)]. Ideally, all uni- or bidimensionally measurable lesions should be measured at each assessment. When multiple lesions are present in any organ, such measurements may not be possible and, under such circumstances, up to 6 representative lesions should be selected, if available.

The term “complete response” (CR) as used herein means a complete disappearance of all clinically detectable malignant disease, determined by 2 assessments at least 4 weeks apart.

The term “partial response” (PR) as used herein means a 50% or greater reduction from baseline in the sum of the products of the longest perpendicular diameters of all measurable disease without progression of evaluable disease and without evidence of any new lesions as determined by at least two consecutive assessments at least four weeks apart. Assessments should show a partial decrease in the size of lytic lesions, recalcifications of lytic lesions, or decreased density of blastic lesions.

The term “advanced glycation end product receptor 1” or “AGER1”, which is also known as “dolichyl-diphosphooligosaccharide-protein glycosyltransferase non-catalytic subunit” or “DDOST” encompasses all forms of AGER1 and also includes biologically active fragments, variants, analogs, and derivatives thereof that retain biological activity (e.g., binding to AGEs and/or detoxification of AGEs). An AGER1 polynucleotide, nucleic acid, oligonucleotide, protein, polypeptide, or peptide refers to a molecule derived from any source. The molecule need not be physically derived from an organism, but may be synthetically or recombinantly produced. A number of AGER1 nucleic acid and protein sequences are known. A representative sequence of a human AGER1 protein is presented in SEQ ID NO:1. Additional representative sequences are listed in the National Center for Biotechnology Information (NCBI) database. See, for example, NCBI entries: Accession Nos. NG_032064, NM_005216, NM_001003321, NM_001168008, NM_007838, AB012717, NM_001101073, XM_001161661, NP_005207, EAW94937, EAW94936, EAW94940, EAW94939, AAH02594, XP_001161661, JAA43906, JAA43905, O54734, NP_031864, BAB82434, EDL13259, EDL13258, and EDL13257; all of which sequences (as entered by the date of filing of this application) are herein incorporated by reference. Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to produce an AGER1 protein or recombinant polynucleotide comprising a coding sequence encoding an AGER1 protein for use in the methods described herein.

By “fragment” is intended a molecule consisting of only a part of the intact full-length sequence and structure. The fragment can include a C-terminal deletion an N-terminal deletion, and/or an internal deletion of the polypeptide. Active fragments of a particular protein or polypeptide will generally include at least about 5-14 contiguous amino acid residues of the full length molecule, but may include at least about 15-25 contiguous amino acid residues of the full length molecule, and can include at least about 20-50 or more contiguous amino acid residues of the full length molecule, or any integer between 5 amino acids and the full length sequence, provided that the fragment in question retains biological activity (e.g., AGER1 biological activity) or anti-cancer activity.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

“Active molecule” or “active agent” as described herein includes any agent, drug, compound, composition of matter or mixture which provides some pharmacologic, often beneficial, effect that can be demonstrated in vivo or in vitro. This includes foods, food supplements, nutrients, nutriceuticals, drugs, vaccines, antibodies, vitamins, and other beneficial agents. As used herein, the terms further include any physiologically or pharmacologically active substance that produces a localized or systemic effect in a patient.

“Substantially” or “essentially” means nearly totally or completely, for instance, 95% or greater of some given quantity.

“Substantially purified” generally refers to isolation of a substance (e.g., compound, small molecule, drug, polynucleotide, protein, polypeptide, antibody, aptamer, AGE inhibitor or crosslink breaker) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

“Isolated” refers to an entity of interest that is in an environment different from that in which it may naturally occur. “Isolated” is meant to include entities that are within samples that are substantially enriched for the entity of interest and/or in which the entity of interest is partially or substantially purified.

“Diagnosis” as used herein generally includes determination as to whether a subject is likely affected by a given disease, disorder or dysfunction. The skilled artisan often makes a diagnosis on the basis of one or more diagnostic indicators, e.g., viscoelasticity, which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with HCC) as well as those in which prevention is desired (e.g., those with a genetic predisposition to developing HCC, those with a disease or condition that increases susceptibility to developing HCC (e.g., cirrhosis of the liver, nonalcoholic steatohepatitis, type 2 diabetes mellitus, viral hepatitis (e.g., hepatitis B, hepatitis C), alpha 1-antitrypsin deficiency, Wilson's disease, alcoholism), those with an environmental exposure to a carcinogen or toxin (e.g., aflatoxin, pyrrolizidine alkaloids, or hemochromatosis), or who otherwise have an increased susceptibility or increased likelihood of developing HCC, those suspected of having HCC (e.g., abnormal blood tests (alpha-fetoprotein and des-gamma carboxyprothrombin levels), etc.).

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

The term “about,” particularly in reference to a given quantity, is meant to encompass deviations of plus or minus five percent.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

A “therapeutically effective dose” or “therapeutic dose” is an amount sufficient to effect desired clinical results (i.e., achieve therapeutic efficacy). A therapeutically effective dose can be administered in one or more administrations.

“Providing an analysis” is used herein to refer to the delivery of an oral or written analysis (i.e., a document, a report, etc.). A written analysis can be a printed or electronic document. A suitable analysis (e.g., an oral or written report) provides any or all of the following information: identifying information of the subject (name, age, etc.), a description of the technique used to measure the viscoelasticity of liver tissue (e.g., atomic force microscopy, magnetic resonance elastography, or ultrasound), the results of the assay, the assessment as to whether the individual is determined to have hepatocellular carcinoma or be at risk of developing hepatocellular carcinoma, a recommendation for treatment (e.g., a particular anti-cancer therapy), and/or to continue or alter therapy, a recommended strategy for additional therapy, etc. The report can be in any format including, but not limited to printed information on a suitable medium or substrate (e.g., paper); or electronic format. If in electronic format, the report can be in any computer readable medium, e.g., diskette, compact disk (CD), flash drive, and the like, on which the information has been recorded. In addition, the report may be present as a website address which may be used via the internet to access the information at a remote site.

“Homology” refers to the percent identity between two polynucleotide or two polypeptide molecules. Two nucleic acid, or two polypeptide sequences are “substantially homologous” to each other when the sequences exhibit at least about 50% sequence identity, preferably at least about 75% sequence identity, more preferably at least about 80% 85% sequence identity, more preferably at least about 90% sequence identity, and most preferably at least about 95% 98% sequence identity over a defined length of the molecules. As used herein, substantially homologous also refers to sequences showing complete identity to the specified sequence.

In general, “identity” refers to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M.O. in Atlas of Protein Sequence and Structure M.O. Dayhoff ed., 5 Suppl. 3:353 358, National biomedical Research Foundation, Washington, DC, which adapts the local homology algorithm of Smith and Waterman Advances in Appl. Math. 2:482 489, 1981 for peptide analysis. Programs for determining nucleotide sequence identity are available in the Wisconsin Sequence Analysis Package, Version 8 (available from Genetics Computer Group, Madison, WI) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, CA). From this suite of packages, the Smith Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single stranded specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

“Recombinant” as used herein to describe a nucleic acid molecule means a polynucleotide of genomic, cDNA, viral, semisynthetic, or synthetic origin which, by virtue of its origin or manipulation, is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Recombinant host cells,” “host cells,” “cells”, “cell lines,” “cell cultures,” and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence.

“Expression cassette” or “expression construct” refers to an assembly which is capable of directing the expression of the sequence(s) or gene(s) of interest. An expression cassette generally includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include, one or more selectable markers, a signal which allows the plasmid construct to exist as single stranded DNA (e.g., a M13 origin of replication), at least one multiple cloning site, and a “mammalian” origin of replication (e.g., a SV40 or adenovirus origin of replication).

“Purified polynucleotide” refers to a polynucleotide of interest or fragment thereof which is essentially free, e.g., contains less than about 50%, preferably less than about 70%, and more preferably less than about at least 90%, of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density.

The term “transfection” is used to refer to the uptake of foreign DNA by a cell. A cell has been “transfected” when exogenous DNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA moieties into suitable host cells. The term refers to both stable and transient uptake of the genetic material, and includes uptake of peptide-or antibody-linked DNAs.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

The terms “variant,” “analog” and “mutein” refer to biologically active derivatives of the reference molecule that retain desired activity, such as AGER1 activity or anti-cancer activity. In general, the terms “variant” and “analog” refer to compounds having a native polypeptide sequence and structure with one or more amino acid additions, substitutions (generally conservative in nature) and/or deletions, relative to the native molecule, so long as the modifications do not destroy biological activity, and which are “substantially homologous” to the reference molecule as defined below. In general, the amino acid sequences of such analogs will have a high degree of sequence homology to the reference sequence, e.g., amino acid sequence homology of more than 50%, generally more than 60%-70%, even more particularly 80%-85% or more, such as at least 90%-95% or more, when the two sequences are aligned. Often, the analogs will include the same number of amino acids but will include substitutions, as explained herein. The term “mutein” further includes polypeptides having one or more amino acid-like molecules including but not limited to compounds comprising only amino and/or imino molecules, polypeptides containing one or more analogs of an amino acid (including, for example, unnatural amino acids, etc.), polypeptides with substituted linkages, as well as other modifications known in the art, both naturally occurring and non-naturally occurring (e.g., synthetic), cyclized, branched molecules and the like. The term also includes molecules comprising one or more N-substituted glycine residues (a “peptoid”) and other synthetic amino acids or peptides. (See, e.g., U.S. Pat. Nos. 5,831,005; 5,877,278; and 5,977,301; Nguyen et al., Chem. Biol. (2000) 7:463-473; and Simon et al., Proc. Natl. Acad. Sci. USA (1992) 89:9367-9371 for descriptions of peptoids). Methods for making polypeptide analogs and muteins are known in the art and are described further below.

As explained above, analogs generally include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, and tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 conservative or non-conservative amino acid substitutions, or any integer between 5-25, so long as the desired function of the molecule remains intact. One of skill in the art may readily determine regions of the molecule of interest that can tolerate change by reference to Hopp/Woods and Kyte-Doolittle plots, well known in the art.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, adenoviruses, lentiviruses, alphaviruses, pox viruses, and vaccinia viruses.

A polynucleotide “derived from” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

### Diagnostic Methods

AGEs in the liver promote HCC in vivo by increasing tissue viscoelasticity. HCC cell spread and migration is associated with increased viscoelasticity. Accordingly, monitoring viscoelasticity of liver tissue is useful for prognosis, diagnosis, therapy selection, and monitoring treatment of HCC. In one aspect, a method of diagnosing HCC in a patient is provided, the method comprising measuring viscoelasticity of liver tissue from the patient, wherein increased viscoelasticity of the liver tissue of the patient compared to reference value ranges for viscoelasticity of liver tissue from a control subject indicates that the patient is at risk of developing HCC or has a positive diagnosis for HCC.

Any suitable method known in the art can be used for measuring viscoelasticity of liver tissue in a subject. Exemplary techniques for measuring viscoelasticity include, but are not limited to, atomic force microscopy, magnetic resonance elastography, and ultrasound. For a description of the use of these methods for measuring viscoelasticity, see, e.g., Krieg et al. (2019) Nature Reviews Physics 1:41-57, Efremov et al. (2020) Soft Matter. 16(1):64-81, Tripathy et al. (2009) J. Biomech. Eng. 131(9):094507, Costa et al. (2003-2004) Dis. Markers 19(2-3):139-154, Xiao et al. (2017) Hepatology 66(5):1486-1501, Li et al. (2020) Magn. Reason. Imaging Clin. N. Am. 28(3):331-340, Hoodeshenas et al. (2018) Top. Magn. Reason. Imaging 27(5):319-333, Li et al. (2017) Proc. Math Phys. Eng. Sci. 473(2199):20160841, Tang et al. (2015) AJR Am. J. Roentgenol. 205(1):22-32, Sigrist et al. (2017) Theranostics 7(5):1303-1329, Ozturk et al. (2018) Abdom Radiol (NY) 43(4):773-78, and Gennisson et al. (2013) Diagn. Interv. Imaging 94(5):487-95; herein incorporated by reference in their entireties.

When analyzing the viscoelasticity of liver tissue of a patient, the reference value ranges used for comparison can represent the values of the viscoelasticity of liver tissue from one or more subjects without HCC (i.e., normal or healthy control), wherein increased viscoelasticity of the liver tissue of the patient compared to the reference value ranges for a normal control subject indicates the patient is at risk of developing HCC or has HCC. Alternatively, the reference value ranges can represent the values of the viscoelasticity of liver tissue from one or more subjects with HCC, wherein similarity to the reference value ranges indicates the patient is at risk of developing HCC or has HCC.

The methods described herein may be used to determine an appropriate treatment regimen for a patient and, in particular, whether a patient should be monitored more frequently for development of HCC or treated for HCC. For example, a patient is selected for treatment for HCC if the patient has a positive diagnosis for HCC based on the measured viscoelasticity, as described herein. In some cases, the diagnostic methods described herein may be used by themselves or combined with medical imaging to confirm the diagnosis and further evaluate the extent of cancerous disease (how far and where the cancer has spread) to aid in determining prognosis and evaluating optimal strategies for treatment (e.g., surgery, radionuclide therapy, chemotherapy, targeted therapy, immunotherapy, biologic therapy, etc.). Exemplary medical imaging techniques include, without limitation, magnetic resonance imaging (MRI), positron emission tomography (PET), single photon emission computed tomography (SPECT), computed tomography (CT), ultrasound imaging (UI), optical imaging (OI), photoacoustic imaging (PI), fluoroscopy, and fluorescence imaging.

In some embodiments, the measured viscoelasticity is used in combination with biomarkers for diagnosing HCC, such as alpha-fetoprotein (AFP) or des-gamma carboxyprothrombin (DCP). For example, blood levels of AFP or DCP or a combination thereof can be monitored in addition to measuring viscoelasticity of liver tissue.

Exemplary treatments for HCC include, without limitation, tumor surgical resection, radiofrequency ablation (RFA), cryoablation, percutaneous ethanol or acetic acid injection, transcatheter arterial chemoembolization (TACE), selective internal radiation therapy (SIRT), high intensity focused ultrasound, or external beam therapy, liver transplantation, portal vein embolization, or administering anti-cancer therapeutic agents such as chemotherapeutic agents (e.g., cisplatin, gemcitabine, oxaliplatin, doxorubicin, 5-fluorouracil, capecitabine, or mitoxantrone), targeted therapeutic agents (e.g., sorafenib, regorafenib, lenvatinib, or cabozantinib), immunotherapeutic agents (e.g., ramucirumab, nivolumab, or pembrolizumab), or radioisotopes (e.g., Yttrium-90, Iodine-131, Rhenium-188, or Holmium-166), or a combination thereof.

The viscoelasticity of liver tissue can be monitored in a patient who has HCC or is at risk of developing HCC. For example, the viscoelasticity of liver tissue can be measured at a first time point and a second time point, wherein detection of increased viscoelasticity indicates that the HCC is progressing, and detection of decreased viscoelasticity of liver tissue indicates that the HCC is not progressing. In some embodiments, the patient is monitored over a period of time by repeatedly measuring viscoelasticity of liver tissue to determine whether or not the HCC is progressing.

The subject methods are especially useful for diagnosing or monitoring a patient, as described herein, if the patient has an underlying condition or disease that makes the patient susceptible to developing HCC. Exemplary conditions and diseases that increase susceptibility to HCC include, but are not limited to, liver inflammation, traumatic injury to the liver, liver cirrhosis, fatty liver disease, hepatitis (e.g., alcoholic hepatitis, non-alcoholic steatohepatitis, autoimmune hepatitis, drug-induced hepatitis, or viral hepatitis), a hepatitis A virus infection, a hepatitis B virus infection, a hepatitis C virus infection, a hepatitis D virus infection, a hepatitis E virus infection, type 2 diabetes mellitus, type 2 diabetes mellitus combined with non-alcoholic steatohepatitis, hereditary hemochromatosis, Wilson disease, primary biliary cirrhosis, and α-1-antitrypsin deficiency.

The subject methods may also be used for monitoring a patient before and after treatment for HCC to determine whether the patient is responsive or not responsive to the treatment. For example, the viscoelasticity of the liver tissue can be measured before the subject undergoes the therapy and after the subject undergoes the therapy, wherein detection of increased viscoelasticity of the liver tissue after the treatment compared to before the treatment indicates that the HCC is progressing or not responding to the treatment, and detection of decreased viscoelasticity of the liver tissue after the treatment compared to before the treatment indicates that the HCC is not progressing.

The viscoelasticity of the liver tissue before treatment can be referred to as a “pre-treatment value” because the first measurement of the viscoelasticity of the liver tissue is prior to the administration of the therapy (i.e., “pre-treatment”). The pre-treatment viscoelasticity value can also be referred to as a “baseline value” because this value is the value to which “post-treatment” values are compared. In some cases, the baseline value (i.e., “pre-treatment value”) is determined by determining the viscoelasticity of the liver tissue multiple times (i.e., more than one, e.g., two or more, three or more, four or more, five or more, etc.). In some cases, the multiple measurements of the viscoelasticity of the liver tissue are made at different time points in order to assess natural fluctuations in viscoelasticity values prior to treatment. As such, in some cases, one or more (e.g., two or more, three or more, four or more, five or more, etc. pre-treatment measurements are made of the viscoelasticity of the liver tissue). In some cases, the viscoelasticity of the liver tissue is determined at least twice and a “pre-treatment value” is calculated by averaging the separate measurements.

The viscoelasticity of the liver tissue is also measured after the administration of a therapy. Thus, the viscoelasticity of the liver tissue after treatment can be referred to as a “post-treatment value”. In some embodiments, the viscoelasticity of the liver tissue is measured at multiple times after the treatment (e.g., two or more, three or more, four or more, five or more, etc. post-treatment measurements are made of the viscoelasticity of the liver tissue). These measurements of the viscoelasticity of the liver tissue can also be referred to as “post-treatment values.”

The term “responsive” as used herein means that the treatment is having the desired effect such as an anti-tumor effect. For example, a positive therapeutic response would refer to one or more of the following improvements in the disease: (1) reduction in tumor size; (2) reduction in the number of cancer cells; (3) inhibition (i.e., slowing to some extent, preferably halting) of tumor growth; (4) inhibition (i.e., slowing to some extent, preferably halting) of cancer cell infiltration into peripheral organs; (5) inhibition (i.e., slowing to some extent, preferably halting) of tumor metastasis; and (6) some extent of relief from one or more symptoms associated with the cancer. When the individual does not improve in response to the treatment, it may be desirable to seek a different therapy or treatment regime for the individual.

The determination that an individual is at risk of developing HCC or has HCC is an active clinical application of the correlation between the viscoelasticity of the liver tissue and the disease. For example, “determining” requires the active step of reviewing the data, which is produced during the active assaying step(s), and resolving whether an individual does or does not have HCC, or is responding or not responding to a therapy for treatment of HCC. Additionally, in some cases, a decision is made to proceed with the current treatment (i.e., therapy), or instead to alter the treatment. In some cases, the subject methods include the step of continuing therapy or altering therapy.

The term “continue treatment” (i.e., continue therapy) is used herein to mean that the current course of treatment (e.g., continued administration of a therapy) is to continue. If the current course of treatment is not effective in reducing liver viscoelasticity, treating HCC, or preventing disease progression, the treatment may be altered. “Altering therapy” is used herein to mean “discontinuing therapy” or “changing the therapy” (e.g., changing the type of treatment, changing the particular dose and/or frequency of administration of medication, e.g., increasing the dose and/or frequency). In some cases, therapy can be altered until the individual is deemed to be responsive. In some embodiments, altering therapy means changing which type of treatment is administered, discontinuing a particular treatment altogether, etc.

As a non-limiting illustrative example, a patient may be initially treated with an AGE inhibitor and/or crosslink breaker. Then to “continue treatment” would be to continue with this type of treatment. If the current course of treatment is not effective, the treatment may be altered, e.g., increasing dosage or frequency of the treatment, or changing to a different treatment. Switching treatment might involve, for example, administering a different AGE inhibitor or crosslink breaker.

In another illustrative example, a patient may be initially treated with a chemotherapeutic agent. Then to “continue treatment” would be to continue with this type of treatment. If the current course of treatment is not effective, the treatment may be altered, e.g., increasing dosage or frequency of a treatment for HCC, changing to a different treatment, or starting palliative care for the patient. Switching treatment might involve, for example, administering a different chemotherapeutic agent or administering a different type of anti-cancer therapy such as surgery, radiation therapy, immunotherapy, etc.

In other words, the viscoelasticity of the liver tissue may be monitored in order to determine when to continue therapy and/or when to alter therapy. As such, a post-treatment value of the viscoelasticity of the liver tissue is used to determine whether an individual being treated for HCC, is responsive or is maintaining responsiveness to a treatment.

The therapy can be administered to an individual any time after a pre-treatment values for the viscoelasticity of the liver tissue is measured for the individual, but it is preferable for the therapy to be administered simultaneous with or as soon as possible (e.g., about 7 days or less, about 3 days or less, e.g., 2 days or less, 36 hours or less, 1 day or less, 20 hours or less, 18 hours or less, 12 hours or less, 9 hours or less, 6 hours or less, 3 hours or less, 2.5 hours or less, 2 hours or less, 1.5 hours or less, 1 hour or less, 45 minutes or less, 30 minutes or less, 20 minutes or less, 15 minutes or less, 10 minutes or less, 5 minutes or less, 2 minutes or less, or 1 minute or less) after a pre-treatment value for the viscoelasticity of the liver tissue is measured (or, when multiple pre-treatment measurements of the viscoelasticity are made, after the final measurement of the viscoelasticity).

In some cases, more than one type of therapy may be administered to the individual. For example, a subject who has HCC may undergo surgical resection of a tumor followed by administration of a chemotherapeutic agent or biologic agent. Systemic therapy may be administered if the cancer spreads beyond the site of the primary tumor or undergoes metastasis.

In some embodiments, the viscoelasticity of the liver tissue is used for monitoring for a recurrence of HCC in a patient. For example, a first value of the viscoelasticity of the liver tissue is measured after treatment for a previous occurrence of HCC at a first time point when the patient is characterized as cancer-free from imaging or other diagnostic modalities. A second value of the viscoelasticity of the liver tissue is measured at a second time point during a period of monitoring for the recurrence. If the patient has a positive diagnosis for the recurrence of the HCC based on the value of the viscoelasticity of the liver tissue, the patient should be treated for the recurrence of the HCC. In some embodiments, the patient is monitored for a recurrence over a period of time by repeatedly measuring the viscoelasticity of the liver tissue to determine whether or not the patient has a recurrence of HCC or is at increased risk of recurrence of HCC. In some embodiments, the patient is monitored for a recurrence repeatedly over a period of 1 month, 2 months, 4 months, 6 months, 8 months, 1 year, 2 years, 3 years, 4 years, 5 years, or longer by the methods described herein.

In one embodiment, a method of monitoring for a recurrence of HCC in a patient and treating the patient for the recurrence is provided, the method comprising: a) measuring the viscoelasticity of the liver tissue of the patient after treatment of the patient for a previous occurrence of HCC at a first time point when the patient is characterized as cancer-free from imaging or other diagnostic modalities; b) measuring the viscoelasticity of the liver tissue of the patient at a second time point during a period of monitoring for the recurrence, wherein increased viscoelasticity of the liver tissue of the patient at the second time point indicates that the HCC has recurred; c) treating the patient for the recurrence of the HCC, if the patient has a positive diagnosis for the recurrence of the HCC based on measuring the viscoelasticity of the liver tissue of the patient; and f) repeating step b) subsequently during the period of monitoring for the recurrence.

In some embodiments, the subject methods include providing an analysis indicating whether the individual is determined to be at risk of developing HCC or a recurrence of HCC, or has HCC or a recurrence of HCC. The analysis may further provide an analysis of whether an individual is responsive or not responsive to a treatment, or whether the individual is determined to be maintaining responsiveness or not maintaining responsiveness to a treatment for HCC. As described above, an analysis can be an oral or written report (e.g., written or electronic document). The analysis can be provided to the subject, to the subject's physician, to a testing facility, etc. The analysis can also be accessible as a website address via the internet. In some such cases, the analysis can be accessible by multiple different entities (e.g., the subject, the subject's physician, a testing facility, etc.).

### Nucleic Acids Encoding AGER1

Nucleic acids encoding AGER1 can be used to treat HCC. A nucleic acid encoding AGER1can be inserted into an expression vector to create an expression cassette capable of producing the AGER1 in a suitable host cell. The ability of constructs to produce the AGER1 can be empirically determined.

A number of AGER1 nucleic acid and protein sequences are known. A representative sequence of a human AGER1 protein is presented in SEQ ID NO:1. Additional representative sequences are listed in the National Center for Biotechnology Information (NCBI) database. See, for example, NCBI entries: Accession Nos. NG_032064, NM_005216, NM_001003321, NM_001168008, NM_007838, AB012717, NM_001101073, XM_001161661, NP_005207, EAW94937, EAW94936, EAW94940, EAW94939, AAH02594, XP_001161661, JAA43906, JAA43905, O54734, NP_031864, BAB82434, EDL13259, EDL13258, and EDL13257; all of which sequences (as entered by the date of filing of this application) are herein incorporated by reference. Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to produce an AGER1 protein or recombinant polynucleotide comprising a coding sequence encoding an AGER1 protein for use in the methods described herein.

Expression cassettes typically include control elements operably linked to the coding sequence, which allow for the expression of the gene in vivo in the subject species. For example, typical promoters for mammalian cell expression include the SV40 early promoter, a CMV promoter such as the CMV immediate early promoter, the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression. Typically, transcription termination and polyadenylation sequences will also be present, located 3′ to the translation stop codon. Preferably, a sequence for optimization of initiation of translation, located 5′ to the coding sequence, is also present. Examples of transcription terminator/polyadenylation signals include those derived from SV40, as described in Sambrook et al., supra, as well as a bovine growth hormone terminator sequence.

Enhancer elements may also be used herein to increase expression levels of the mammalian constructs. Examples include the SV40 early gene enhancer, as described in Dijkema et al., EMPO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777 and elements derived from human CMV, as described in Boshart et al., Cell (1985) 41:521, such as elements included in the CMV intron A sequence.

Once complete, the constructs encoding AGER1 can be administered to a subject using standard gene delivery protocols. Methods for gene delivery are known in the art. See, e.g., U.S. Pat. Nos. 5,399,346, 5,580,859, 5,589,466. Genes can be delivered either directly to a subject or, alternatively, delivered ex vivo, to cells derived from the subject and the cells reimplanted in the subject.

A number of viral based systems have been developed for gene transfer into mammalian cells. These include adenoviruses, retroviruses (γ-retroviruses and lentiviruses), poxviruses, adeno-associated viruses, baculoviruses, and herpes simplex viruses (see e.g., Warnock et al. (2011) Methods Mol. Biol. 737:1-25; Walther et al. (2000) Drugs 60(2):249-271; and Lundstrom (2003) Trends Biotechnol. 21(3):117-122; herein incorporated by reference).

For example, retroviruses provide a convenient platform for gene delivery systems. Selected sequences can be inserted into a vector and packaged in retroviral particles using techniques known in the art. The recombinant virus can then be isolated and delivered to cells of the subject either in vivo or ex vivo. A number of retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; Boris-Lawrie and Temin (1993) Cur. Opin. Genet. Develop. 3:102-109; and Ferry et al. (2011) Curr Pharm Des. 17(24):2516-2527). Lentiviruses are a class of retroviruses that are particularly useful for delivering polynucleotides to mammalian cells because they are able to infect both dividing and nondividing cells (see e.g., Lois et al (2002) Science 295:868-872; Durand et al. (2011) Viruses 3(2):132-159; herein incorporated by reference).

A number of adenovirus vectors have also been described. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476). Additionally, various adeno-associated virus (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor Laboratory Press); Carter, B. J. Current Opinion in Biotechnology (1992) 3:533-539; Muzyczka, N. Current Topics in Microbiol. and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875.

Another vector system useful for delivering the polynucleotides encoding AGER1 is the enterically administered recombinant poxvirus vaccines described by Small, Jr., P. A., et al. (U.S. Pat. No. 5,676,950, issued Oct. 14, 1997, herein incorporated by reference).

Additional viral vectors which will find use for delivering the nucleic acid molecules encoding the AGER1 include those derived from the pox family of viruses, including vaccinia virus and avian poxvirus. By way of example, vaccinia virus recombinants expressing the AGER1 can be constructed as follows. The DNA encoding the particular AGER1 coding sequence is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the coding sequences of interest into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can also be used to deliver the genes. Recombinant avipox viruses, expressing immunogens from mammalian pathogens, are known to confer protective immunity when administered to non-avian species. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with. respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the Alphavirus genus, such as, but not limited to, vectors derived from the Sindbis virus (SIN), Semliki Forest virus (SFV), and Venezuelan Equine Encephalitis virus (VEE), will also find use as viral vectors for delivering the polynucleotides of the present invention. For a description of Sindbis-virus derived vectors useful for the practice of the instant methods, see, Dubensky et al. (1996) J. Virol. 70:508-519; and International Publication Nos. WO 95/07995, WO 96/17072; as well as Dubensky, Jr., T. W., et al., U.S. Pat. No. 5,843,723, issued Dec. 1, 1998, and Dubensky, Jr., T. W., U.S. Pat. No. 5,789,245, issued Aug. 4, 1998, both herein incorporated by reference. Particularly preferred are chimeric alphavirus vectors comprised of sequences derived from Sindbis virus and Venezuelan equine encephalitis virus. See, e.g., Perri et al. (2003) J. Virol. 77: 10394-10403 and International Publication Nos. WO 02/099035, WO 02/080982, WO 01/81609, and WO 00/61772; herein incorporated by reference in their entireties.

A vaccinia-based infection/transfection system can be conveniently used to provide for inducible, transient expression of the coding sequences of interest (for example, a AGER1 expression cassette) in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide of interest, driven by a T7 promoter. The polymerase expressed in the cytoplasm from the vaccinia virus recombinant transcribes the transfected DNA into RNA which is then translated into protein by the host translational machinery. The method provides for high level, transient, cytoplasmic production of large quantities of RNA and its translation products. See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

As an alternative approach to infection with vaccinia or avipox virus recombinants, or to the delivery of genes using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase which in turn will transcribe more template. Concomitantly, there will be a cDNA whose expression is under the control of the T7 promoter. Thus, some of the T7 RNA polymerase generated from translation of the amplification template RNA will lead to transcription of the desired gene. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase. For a further discussion of T7 systems and their use for transforming cells, see, e.g., International Publication No. WO 94/26911; Studier and Moffatt, J. Mol. Biol. (1986) 189:113-130; Deng and Wolff, Gene (1994) 143:245-249; Gao et al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

The synthetic expression cassette of interest can also be delivered without a viral vector. For example, the synthetic expression cassette can be packaged as DNA or RNA in liposomes prior to delivery to the subject or to cells derived therefrom. Lipid encapsulation is generally accomplished using liposomes which are able to stably bind or entrap and retain nucleic acid. The ratio of condensed DNA to lipid preparation can vary but will generally be around 1:1 (mg DNA:micromoles lipid), or more of lipid. For a review of the use of liposomes as carriers for delivery of nucleic acids, see, Hug and Sleight, Biochim. Biophys. Acta. (1991.) 1097:1-17; Straubinger et al., in Methods of Enzymology (1983), Vol. 101, pp. 512-527.

Liposomal preparations for use in the present invention include cationic (positively charged), anionic (negatively charged) and neutral preparations, with cationic liposomes particularly preferred. Cationic liposomes have been shown to mediate intracellular delivery of plasmid DNA (Felgner et al., Proc. Natl. Acad. Sci. USA (1987) 84:7413-7416); mRNA (Malone et al., Proc. Natl. Acad. Sci. USA (1989) 86:6077-6081); and purified transcription factors (Debs et al., J. Biol. Chem. (1990) 265:10189-10192), in functional form.

Cationic liposomes are readily available. For example, N[1-2,3-dioleyloxy) propyl]-N,N,N-triethylammonium (DOTMA) liposomes are available under the trademark Lipofectin, from GIBCO BRL, Grand Island, N.Y. (See, also, Felgner et al., Proc. Natl. Acad. Sci. USA (1987) 84:7413-7416). Other commercially available lipids include (DDAB/DOPE) and DOTAP/DOPE (Boerhinger). Other cationic liposomes can be prepared from readily available materials using techniques well known in the art. See, e.g., Szoka et al., Proc. Natl. Acad. Sci. USA (1978) 75:4194-4198; PCT Publication No. WO 90/11092 for a description of the synthesis of DOTAP (1,2-bis(oleoyloxy)-3-(trimethylammonio)propane) liposomes.

Similarly, anionic and neutral liposomes are readily available, such as, from Avanti Polar Lipids (Birmingham, AL), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

The liposomes can comprise multilammelar vesicles (MLVs), small unilamellar vesicles (SUVs), or large unilamellar vesicles (LUVs). The various liposome-nucleic acid complexes are prepared using methods known in the art. See, e.g., Straubinger et al., in Methods of Immunology (1983), Vol. 101, pp. 512-527; Szoka et al., Proc. Natl. Acad. Sci. USA (1978) 75:4194-4198; Papahadjopoulos et al., Biochim. Biophys. Acta (1975) 394:483; Wilson et al., Cell (1979) 17:77); Deamer and Bangham, Biochim. Biophys. Acta (1976) 443:629; Ostro et al., Biochem. Biophys. Res. Commun. (1977) 76:836; Fraley et al., Proc. Natl. Acad. Sci. USA (1979) 76:3348); Enoch and Strittmatter, Proc. Natl. Acad. Sci. USA (1979) 76:145); Fraley et al., J. Biol. Chem. (1980) 255:10431; Szoka and Papahadjopoulos, Proc. Natl. Acad. Sci. USA (1978) 75:145; and Schaefer-Ridder et al., Science (1982) 215:166.

The DNA and/or peptide(s) can also be delivered in cochleate lipid compositions similar to those described by Papahadjopoulos et al., Biochem. Biophys. Acta (1975) 394:483-491. See, also, U.S. Pat. Nos. 4,663,161 and 4,871,488.

The expression cassette of interest may also be encapsulated, adsorbed to, or associated with, particulate carriers. Examples of particulate carriers include those derived from polymethyl methacrylate polymers, as well as microparticles derived from poly(lactides) and poly(lactide-co-glycolides), known as PLG. See, e.g., Jeffery et al., Pharm. Res. (1993) 10:362-368; McGee J. P., et al., J Microencapsul. 14(2):197-210, 1997; O'Hagan D. T., et al., Vaccine 11(2):149-54, 1993.

Furthermore, other particulate systems and polymers can be used for the in vivo or ex vivo delivery of the nucleic acid of interest. For example, polymers such as polylysine, polyarginine, polyornithine, spermine, spermidine, as well as conjugates of these molecules, are useful for transferring a nucleic acid of interest. Similarly, DEAE dextran-mediated transfection, calcium phosphate precipitation or precipitation using other insoluble inorganic salts, such as strontium phosphate, aluminum silicates including bentonite and kaolin, chromic oxide, magnesium silicate, talc, and the like, will find use with the present methods. See, e.g., Felgner, P. L., Advanced Drug Delivery Reviews (1990) 5:163-187, for a review of delivery systems useful for gene transfer. Peptoids (Zuckerman, R. N., et al., U.S. Pat. No. 5,831,005, issued Nov. 3, 1998, herein incorporated by reference) may also be used for delivery of a construct of the present invention.

Additionally, biolistic delivery systems employing particulate carriers such as gold and tungsten, are especially useful for delivering synthetic expression cassettes encoding AGER1. The particles are coated with the synthetic expression cassette(s) to be delivered and accelerated to high velocity, generally under a reduced atmosphere, using a gun powder discharge from a “gene gun.” For a description of such techniques, and apparatuses useful therefore, see, e.g., U.S. Pat. Nos. 4,945,050; 5,036,006; 5,100,792; 5,179,022; 5,371,015; and 5,478,744. Also, needle-less injection systems can be used (Davis, H. L., et al, Vaccine 12:1503-1509, 1994; Bioject, Inc., Portland, Oreg.).

Recombinant vectors carrying a synthetic expression cassette encoding AGER1 are formulated into compositions for delivery to a vertebrate subject. These compositions may either be prophylactic (to prevent HCC) or therapeutic (to treat HCC). The compositions will comprise a “therapeutically effective amount” of the nucleic acid of interest such that an amount of the AGER1 protein (or a biologically active fragment thereof) can be produced in vivo having anti-cancer activity in the individual to which it is administered. The exact amount necessary will vary depending on the subject being treated; the age and general condition of the subject to be treated; the severity of the condition being treated; the particular AGER1 protein produced and its mode of administration, among other factors. An appropriate effective amount can be readily determined by one of skill in the art. Thus, a “therapeutically effective amount” will fall in a relatively broad range that can be determined through routine trials.

The compositions will generally include one or more “pharmaceutically acceptable excipients or vehicles” such as water, saline, glycerol, polyethyleneglycol, hyaluronic acid, ethanol, etc. Additionally, auxiliary substances, such as wetting or emulsifying agents, pH buffering substances, surfactants and the like, may be present in such vehicles. Certain facilitators of nucleic acid uptake and/or expression can also be included in the compositions or coadministered.

Once formulated, the compositions can be administered directly to the subject (e.g., as described above) or, alternatively, delivered ex vivo, to cells derived from the subject, using methods such as those described above. For example, methods for the ex vivo delivery and reimplantation of transformed cells into a subject are known in the art and can include, e.g., dextran-mediated transfection, calcium phosphate precipitation, polybrene mediated transfection, lipofectamine and LT-1 mediated transfection, protoplast fusion, electroporation, encapsulation of the polynucleotide(s) in liposomes, and direct microinjection of the DNA into nuclei.

Direct delivery of synthetic expression cassette compositions in vivo will generally be accomplished with or without viral vectors, as described above, by injection using either a conventional syringe, needless devices such as Bioject™ or a gene gun, such as the Accell™ gene delivery system (PowderMed Ltd, Oxford, England).

### Animal Model

A non-human animal model of non-alcoholic steatohepatitis with hydrodynamically induced HCC is also provided. The animal model is created by administering advanced glycation end-products to a non-human animal, wherein liver tissue viscoelasticity increases leading to development of HCC (see Examples). The higher viscoelasticity of the liver tissue treated with AGEs promotes development of HCC as well as HCC cell spread and migration.

The non-human animal treated with AGEs may include mammals such as, but not limited to, rodents (e.g., mice, rats, or guinea pigs), non-human primates (e.g., chimpanzees, macaques, monkeys, or apes), cows, pigs, goats, sheep, horses, etc. The subject animals are useful for screening drugs and candidate agents, including AGE inhibitors and crosslink breakers for treating HCC. Suitable screening methods are described further below.

### Isolated Cancerous Liver Cells

Cancerous liver cells may be isolated from the non-human animal model of non-alcoholic steatohepatitis with hydrodynamically induced HCC. The cancerous cells are isolated using standard procedures. Liver cancer cell lines may be derived from such isolated cells, and immortalized using standard techniques, e.g., through use of viruses. The isolated cancerous liver cells are also useful for screening drugs and candidate agents, including AGE inhibitors and crosslink breakers for treating HCC. Of particular interest are isolated cancerous liver cells having AGE cross-linked ECM proteins and increased ECM viscoelasticity for screening for AGE inhibitors and crosslink breakers.

### Screening Candidate Agents for Treatment of HCC

The inventors have discovered that AGEs in the liver promote development of HCC by increasing tissue viscoelasticity (see Examples). Viscoelasticity may be modulated by targeting AGE deposition and crosslinking of ECM proteins such as collagen. Therefore, AGE inhibitors, which can reduce formation of AGEs, deposition of AGEs in the liver, and/or sequester or remove AGEs; and crosslink breakers, which can cleave covalent AGE crosslinks to reduce the level of AGE cross-linked proteins, nucleic acids, and lipids and reduce AGE-mediated toxicity may be useful in treating HCC. Accordingly, screening methods for identifying candidate agents, particularly AGE inhibitors and crosslink breakers that have anti-tumor activity against HCC are provided.

In certain embodiments, candidate agents are tested in a non-human animal model of non-alcoholic steatohepatitis with hydrodynamically induced HCC. For example, a candidate agent can be administered to a non-human animal to determine if an agent has anti-AGE or anti-cancer activity. A variety of assays may be used for this purpose, and in many embodiments, a candidate agent will be tested in different assays to confirm, e.g., inhibition of AGE formation/deposition or cleavage of AGE crosslinks as well as efficacy in treating HCC. Assays may include measurements of the viscoelasticity of liver tissue and/or levels of AGEs and/or AGE-crosslinked proteins, nucleic acids, or lipids in liver tissue in the non-human animal. Viscoelasticity can be measured using methods well known the art, such as, but not limited to, atomic force microscopy, magnetic resonance elastography, and ultrasound.

In other embodiments, cancerous liver cells isolated from the non-human animal model of non-alcoholic steatohepatitis with hydrodynamically induced HCC may are used in assays. For purposes of the cellular assay methods, AGEs and AGE-crosslinked proteins, nucleic acids, and lipids are present in the context of a cell. Any convenient format may be used for the cellular assay, e.g., using wells, plates, flasks, etc., preferably a high throughput format, such as using multi-well plates. Cells are contacted with a test agent of interest and the effect of the agent on ECM viscoelasticity and/or AGE levels, and/or levels of AGE cross-linked proteins, and/or AGE cross-linked nucleic acids, and/or AGE cross-linked lipids can be determined. In addition, cell-based assays may be used, for example, for testing for growth, proliferation of HCC cells in the absence or presence of a candidate agent.

Assays may further include suitable controls (e.g., an animal or cell in the absence of the test agent). Generally, a plurality of assays are run in parallel with different agent concentrations to obtain a differential response to the various concentrations. Typically, one of these concentrations serves as a negative control, i.e., at zero concentration or below the level of detection.

A variety of other reagents may be included in the screening assay. These include reagents like salts, neutral proteins, e.g. albumin, detergents, etc., including agents that are used to facilitate optimal binding activity and/or reduce non-specific or background activity. Reagents that improve the efficiency of the assay, such as protease inhibitors, nuclease inhibitors, anti-microbial agents, etc. may be used. The components of the assay mixture are added in any order that provides for the requisite activity. Incubations are performed at any suitable temperature, typically between 4□C and 40□C. Incubation periods are selected for optimum activity but may also be optimized to facilitate rapid high-throughput screening. In some embodiments, between 0.1 hour and 1 hour, between 1 hour and 2 hours, or between 2 hours and 4 hours, will be sufficient.

A variety of different test agents may be screened. Candidate agents encompass numerous chemical classes, e.g., small organic compounds having a molecular weight of more than 50 daltons and less than about 10,000 daltons, less than about 5,000 daltons, or less than about 2,500 daltons. Test agents can comprise functional groups necessary for structural interaction with proteins, e.g., hydrogen bonding, and can include at least an amine, carbonyl, hydroxyl or carboxyl group, or at least two of the functional chemical groups. The test agents can comprise cyclical carbon or heterocyclic structures and/or aromatic or polyaromatic structures substituted with one or more of the above functional groups. Test agents are also found among biomolecules including peptides, saccharides, fatty acids, steroids, purines, pyrimidines, derivatives, structural analogs or combinations thereof.

Test agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Moreover, screening may be directed to known pharmacologically active compounds and chemical analogs thereof, or to new agents with unknown properties such as those created through rational drug design.

In some embodiments, test agents are synthetic compounds. A number of techniques are available for the random and directed synthesis of a wide variety of organic compounds and biomolecules, including expression of randomized oligonucleotides. See for example WO 94/24314, hereby expressly incorporated by reference, which discusses methods for generating new compounds, including random chemistry methods as well as enzymatic methods.

In another embodiment, the test agents are provided as libraries of natural compounds in the form of bacterial, fungal, plant and animal extracts that are available or readily produced. Additionally, natural or synthetically produced libraries and compounds are readily modified through conventional chemical, physical and biochemical means. Known pharmacological agents may be subjected to directed or random chemical modifications, including enzymatic modifications, to produce structural analogs.

In some embodiments, the test agents are organic moieties. In this embodiment, test agents are synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or candidate agents which can then be tested using the present invention.

In some embodiments test agents are assessed for any cytotoxic activity it may exhibit toward a living eukaryotic cell, using well-known assays, such as trypan blue dye exclusion, an MTT (3-(4,5-dimethylthiazol-2-yl)-2,5-diphenyl-2H-tetrazolium bromide) assay, and the like. Agents that do not exhibit significant cytotoxic activity are considered candidate agents.

In some embodiments, a candidate agent is an AGE inhibitor that reduces formation of AGES, sequesters or removes AGEs, or reduces AGE-mediated toxicity. In some embodiments, an AGE inhibitor may reduce the level of an AGE in the liver by 10%, 20%, 30%, 40%, 50%, 60%, 70%, 80%, 90%, 100%, or any amount in between as compared to native or control levels.

In some embodiments, a candidate agent is a crosslink breaker that cleaves covalent AGE crosslinks between proteins, nucleic acids, or lipids. In some embodiments, a crosslink breaker reduces the level of AGE cross-linked proteins, AGE cross-linked nucleic acids, or AGE cross-linked lipids by 10%, 20%, 30%, 40%, 50%, 60%, 70%, 80%, 90%, 100%, or any amount in between as compared to native or control levels.

In some embodiments, a candidate agent increases expression or activity of AGER1. In some embodiments, the candidate agent increases expression or activity of AGER1 at least 1.5-fold, at least 2-fold, at least 3-fold, at least 4-fold, at least 5-fold, at least 10-fold, at least 20-fold, at least 50-fold, or more as compared to native or control levels.

### Screening Inhibitors for Their Effects on HCC

In some embodiments, a test agent is further tested for its ability to inhibit growth of HCC cells in a cell-based assay. In these embodiments, a test agent of interest is contacted with the HCC cells; and the effect, if any, of the test agent on the HCC cells is determined.

For example, a population of HCC cells can be cultured in vitro in the presence of an effective dose of the test agent. The effect on growth or proliferation may be assayed. The test agent is added to the culture medium, and the culture medium is maintained under conventional conditions suitable for growth of the HCC cells. Various commercially available systems have been developed for the growth of mammalian cells to provide for removal of adverse metabolic products, replenishment of nutrients, and maintenance of oxygen. By employing these systems, the medium may be maintained as a continuous medium, so that the concentrations of the various ingredients are maintained relatively constant or within a prescribed range.

In some embodiments, a test compound is further tested for its efficacy in treating HCC in vivo, e.g., in an animal, such as an animal model of non-alcoholic steatohepatitis with hydrodynamically induced hepatocellular carcinoma, produced as described herein. For example, an agent identified as an AGE inhibitor, crosslink breaker, or as increasing expression or activity of AGER1 can be further tested in an animal model to determine the efficacy, toxicity, or side effects of treatment with such an agent. Monitoring the efficacy of agents (e.g., drugs) for treatment of HCC can be applied not only in basic drug screening, but also in clinical trials. Furthermore, this disclosure pertains to uses of novel agents identified as having anti-cancer activity by the above-described screening assays for treatment of HCC.

### Pharmaceutical Compositions

An AGE inhibitor and/or crosslink breaker that cleaves crosslinks formed by an AGE and/or a recombinant polynucleotide comprising a coding sequence encoding a AGER1 protein can be formulated into pharmaceutical compositions optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition can also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the AGE inhibitor and/or crosslink breaker and/or a recombinant polynucleotide comprising a coding sequence encoding a AGER1 protein, or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, New Jersey); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the AGE inhibitor and/or crosslink breaker and/or a recombinant polynucleotide comprising a coding sequence encoding a AGER1 protein (e.g., when contained in a drug delivery system) in the composition will vary depending on a number of factors but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, NJ (1998), and Kibbe, A.H., Handbook of Pharmaceutical Excipients, 3rd Edition, American Pharmaceutical Association, Washington, D.C., 2000.

The compositions encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for oral, ocular, or localized delivery.

The pharmaceutical preparations herein can also be housed in a syringe, an implantation device, or the like, depending upon the intended mode of delivery and use. Preferably, the compositions comprising one or more AGE inhibitors and/or crosslink breakers and/or a recombinant polynucleotide comprising a coding sequence encoding a AGER1 protein are in unit dosage form, meaning an amount of a conjugate or composition of the invention appropriate for a single dose, in a premeasured or pre-packaged form.

The compositions herein may optionally include one or more additional agents, such as one or more other drugs for treating hepatocellular carcinoma or other medications. For example, compounded preparations may include at least one AGE inhibitor and/or crosslink breaker and/or a recombinant polynucleotide comprising a coding sequence encoding a AGER1 protein and/or one or more other drugs for treating hepatocellular carcinoma such as chemotherapeutic agents (e.g., cisplatin, gemcitabine, oxaliplatin, doxorubicin, 5-fluorouracil, capecitabine, or mitoxantrone), targeted therapeutic agents (e.g., sorafenib, regorafenib, lenvatinib, or cabozantinib), immunotherapeutic agents (e.g., ramucirumab, nivolumab, or pembrolizumab), or radioisotopes (e.g., Yttrium-90, Iodine-131, Rhenium-188, or Holmium-166); or other drugs for treating hepatocellular carcinoma, or other medications used to treat a subject for a condition or disease. Alternatively, such agents can be contained in a separate composition from the composition comprising the AGE inhibitor and/or crosslink breaker and co-administered concurrently, before, or after the composition comprising the AGE inhibitor and/or crosslink breaker and/or a recombinant polynucleotide comprising a coding sequence encoding a AGER1 protein.

### Administration

At least one therapeutically effective cycle of treatment with an AGE inhibitor (e.g., pyridoxamine), a crosslink breaker that cleaves crosslinks formed by an AGE (e.g., alagebrium), or a combination thereof will be administered to a subject for treatment of hepatocellular carcinoma (HCC). By “therapeutically effective dose or amount” of an AGE inhibitor and/or a crosslink breaker that cleaves crosslinks formed by an AGE is intended an amount that when administered brings about a positive therapeutic response with respect to treatment of an individual for HCC. Of particular interest is an amount of an AGE inhibitor and/or a crosslink breaker that provides an anti-tumor effect, as defined herein. By “positive therapeutic response” is intended the individual undergoing the treatment according to the invention exhibits an improvement in one or more symptoms of the HCC for which the individual is undergoing therapy.

Thus, for example, a “positive therapeutic response” would be an improvement in the disease in association with the therapy, and/or an improvement in one or more symptoms of the disease in association with the therapy. Therefore, for example, a positive therapeutic response would refer to one or more of the following improvements in the disease: (1) reduction in tumor size; (2) reduction in the number of metastatic cancer cells; (3) inhibition (i.e., slowing to some extent, preferably halting) of tumor growth; (4) inhibition (i.e., slowing to some extent, preferably halting) of cancer cell infiltration into peripheral organs; (5) inhibition (i.e., slowing to some extent, preferably halting) of tumor metastasis; and (6) some extent of relief from one or more symptoms associated with the cancer. Such therapeutic responses may be further characterized as to degree of improvement. Thus, for example, an improvement may be characterized as a complete response. By “complete response” is documentation of the disappearance of all symptoms and signs of all measurable or evaluable disease confirmed by physical examination, laboratory, ultrasound, nuclear, radiographic studies (i.e., CT (computer tomography), and/or MRI (magnetic resonance imaging)), and other non-invasive procedures repeated for all initial abnormalities or sites positive at the time of entry into the study. Alternatively, an improvement in the disease may be categorized as being a partial response. By “partial response” is intended a reduction of greater than 50% in the sum of the products of the perpendicular diameters of all measurable lesions when compared with pretreatment measurements.

In certain embodiments, multiple therapeutically effective doses of compositions comprising an AGE inhibitor, and/or a crosslink breaker, and/or one or more other therapeutic agents, such as other drugs for treating cancer, or other medications will be administered according to a daily dosing regimen, or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, and so forth. For example, in some embodiments, an AGE inhibitor and/or a crosslink breaker will be administered twice-weekly or thrice-weekly for an extended period of time, such as for 1, 2, 3, 4, 5, 6, 7, 8 . . . 10 . . . 15 . . . 24 weeks, and so forth. By “twice-weekly” or “two times per week” is intended that two therapeutically effective doses of the agent in question is administered to the subject within a 7-day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7-day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy (i.e., twice-weekly or thrice-weekly administration of a therapeutically effective dose) for one or more weekly cycles until the desired therapeutic response is achieved. The agents can be administered by any acceptable route of administration as noted herein below.

The compositions comprising an AGE inhibitor and/or a crosslink breaker are typically, although not necessarily, administered orally, via injection (subcutaneously, intravenously, or intramuscularly), by infusion, or locally. Additional modes of administration are also contemplated, such as intra-arterial, percutaneous, intraperitoneal, pulmonary, intralesion, transdermal, transmucosal, and so forth. When administering the AGE inhibitor and/or crosslink breaker by injection, the administration may be by continuous infusion or by single or multiple boluses.

The preparations according to the invention are also suitable for local treatment. In a particular embodiment, a composition of the invention is used for localized delivery of an AGE inhibitor and/or a crosslink breaker for the treatment of HCC. For example, compositions may be administered directly into a liver tumor or cancerous liver cells. Administration may be by perfusion through a regional catheter or direct intralesional injection.

The pharmaceutical preparation can be in the form of a liquid solution or suspension immediately prior to administration, but may also take another form such as a syrup, cream, ointment, tablet, capsule, powder, gel, matrix, suppository, or the like. The pharmaceutical compositions comprising the AGE inhibitor, and/or crosslink breaker, and other agents may be administered using the same or different routes of administration in accordance with any medically acceptable method known in the art.

In another embodiment, the pharmaceutical compositions comprising the AGE inhibitor, and/or crosslink breaker, and/or other agents are administered prophylactically, e.g., to prevent cancer progression, cancer recurrence, or metastasis in tissue. Such prophylactic uses will be of particular value for subjects at high risk of cancer recurrence or metastasis.

In another embodiment of the invention, the pharmaceutical compositions comprising the AGE inhibitor, and/or crosslink breaker, and/or other agents are in a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

The invention also provides a method for administering a conjugate comprising an AGE inhibitor and/or a crosslink breaker (e.g., conjugated to a diagnostic or therapeutic agent) as provided herein to a patient suffering from HCC. The method comprises administering, via any of the herein described modes, a therapeutically effective amount of the conjugate or drug delivery system, preferably provided as part of a pharmaceutical composition. The method of administering may be used to treat HCC that is responsive to treatment with an AGE inhibitor and/or a crosslink breaker. In some embodiments, the compositions herein are effective in treating HCC in a subject who also has non-alcoholic steatohepatitis or type 2 diabetes mellitus.

Those of ordinary skill in the art will appreciate which conditions an AGE inhibitor and/or a crosslink breaker can effectively treat. The actual dose to be administered will vary depending upon the age, weight, and general condition of the subject as well as the severity of the condition being treated, the judgment of the health care professional, and conjugate being administered. Therapeutically effective amounts can be determined by those skilled in the art, and will be adjusted to the particular requirements of each particular case.

Generally, a therapeutically effective amount will range from about 0.50 mg to 5 grams of an AGE inhibitor and/or a crosslink breaker daily, more preferably from about 5 mg to 2 grams daily, even more preferably from about 7 mg to 1.5 grams daily. Preferably, such doses are in the range of 10-600 mg four times a day (QID), 200-500 mg QID, 25-600 mg three times a day (TID), 25-50 mg TID, 50-100 mg TID, 50-200 mg TID, 300-600 mg TID, 200-400 mg TID, 200-600 mg TID, 100 to 700 mg twice daily (BID), 100-600 mg BID, 200-500 mg BID, or 200-300 mg BID. The amount of compound administered will depend on the potency of the specific AGE inhibitor and/or crosslink breaker and the magnitude or effect desired and the route of administration.

A purified AGE inhibitor and/or crosslink breaker (again, preferably provided as part of a pharmaceutical preparation) can be administered alone or in combination with one or more other anti-cancer therapeutic agents, such as chemotherapy, immunotherapy, biologic or targeted therapy agents, or other medications used to treat a particular condition or disease according to a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Preferred compositions are those requiring dosing no more than once a day.

An AGE inhibitor can be administered prior to, concurrent with, or subsequent to a crosslink breaker and/or other agents. If provided at the same time as other agents, the AGE inhibitor can be provided in the same or in a different composition. Thus, the AGE inhibitor and crosslink breaker and/or other agents can be presented to the individual by way of concurrent therapy. By “concurrent therapy” is intended administration to a subject such that the therapeutic effect of the combination of the substances is caused in the subject undergoing therapy. For example, concurrent therapy may be achieved by administering a dose of a pharmaceutical composition comprising an AGE inhibitor and a dose of a pharmaceutical composition comprising a crosslink breaker and/or another agent, such as another drug for treating HCC, which in combination comprise a therapeutically effective dose, according to a particular dosing regimen. Similarly, the AGE inhibitor, crosslink breaker, and/or other therapeutic agents can be administered in at least one therapeutic dose. Administration of the separate pharmaceutical compositions can be performed simultaneously or at different times (i.e., sequentially, in either order, on the same day, or on different days), as long as the therapeutic effect of the combination of these substances is caused in the subject undergoing therapy.

Where a subject undergoing therapy in accordance with the previously mentioned dosing regimens exhibits a partial response or a relapse following a prolonged period of remission, subsequent courses of concurrent therapy may be needed to achieve complete remission of the disease. Thus, subsequent to a period of time off from a first treatment period, a subject may receive one or more additional treatment periods with an AGE inhibitor and/or a crosslink breaker. Such a period of time off between treatment periods is referred to herein as a time period of discontinuance. It is recognized that the length of the time period of discontinuance is dependent upon the degree of tumor response (i.e., complete versus partial) achieved with any prior treatment periods of concurrent therapy with these therapeutic agents.

Additionally, treatment with an AGE inhibitor and/or a crosslink breaker may be combined with any other medical treatment for HCC, such as, but not limited to, surgery, radiation therapy, chemotherapy, hormonal therapy, immunotherapy, or molecularly targeted or biologic therapy. Exemplary treatments for HCC include, without limitation, tumor surgical resection, radiofrequency ablation (RFA), cryoablation, percutaneous ethanol or acetic acid injection, transcatheter arterial chemoembolization (TACE), selective internal radiation therapy (SIRT), high intensity focused ultrasound, or external beam therapy, liver transplantation, portal vein embolization, or administering anti-cancer

### Kits

Also provided are kits comprising any of the compositions, animal models, or cells, described herein. In certain embodiments, the kit comprises an AGE inhibitor (e.g., pyridoxamine) and/or a crosslink breaker (e.g., alagebrium). In certain embodiments, the kit comprises a non-human animal model of non-alcoholic steatohepatitis with hydrodynamically induced hepatocellular carcinoma, as described herein. In certain embodiments, the kit comprises a viral vector comprising a coding sequence encoding a AGER1 protein. In some embodiments, the kit comprises an AAV vector comprising a coding sequence encoding a AGER1 protein.

Kits may comprise one or more containers of the compositions described herein. Compositions comprising an AGE inhibitor (e.g., pyridoxamine) and/or a crosslink breaker (e.g., alagebrium) and/or a viral vector comprising a coding sequence encoding a AGER1 protein can be in liquid form or can be lyophilized. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle). The kit can further comprise a container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery device. The kit may also provide a delivery device pre-filled with an AGE inhibitor and/or crosslink breaker.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, Blu-ray, flash drive, and the like, on which the information has been recorded. Yet another form of these instructions that may be present is a website address which may be used via the internet to access the information at a removed site.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-45 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.
1. A method of diagnosing and treating hepatocellular carcinoma (HCC) in a patient, the method comprising:
measuring viscoelasticity of liver tissue from the patient, wherein increased viscoelasticity of the liver tissue of the patient compared to reference value ranges for viscoelasticity of liver tissue from a control subject indicates that the patient is at risk of developing the HCC or has a positive diagnosis for the HCC; and
treating the patient for the HCC, if the patient has a positive diagnosis for the HCC based on the measured viscoelasticity of the liver tissue.
2. The method of aspect 1, wherein the patient has non-alcoholic steatohepatitis or type 2 diabetes mellitus, or a combination thereof.
3. The method of aspect 1 or 2, wherein the liver is cirrhotic or non-cirrhotic.
4. The method of any one of aspects 1-3, wherein the viscoelasticity is measured using atomic force microscopy, magnetic resonance elastography, or ultrasound.
5. The method of any one of aspects 1-4, wherein said treating the patient for HCC comprises surgical resection of an HCC tumor, liver transplantation, radiofrequency ablation, cryoablation, radiation therapy, chemotherapy, immunotherapy, or biologic therapy, or a combination thereof.
6. The method of any one of aspects 1-5, further comprising performing medical imaging of the liver to confirm the positive diagnosis for the HCC.
7. The method of aspect 6, wherein the medical imaging comprises magnetic resonance imaging (MRI), positron emission tomography (PET), single photon emission computed tomography (SPECT), computed tomography (CT), ultrasound imaging (UI), optical imaging (OI), photoacoustic imaging (PI), fluoroscopy, or fluorescence imaging.
8. A method of diagnosing hepatocellular carcinoma (HCC) in a patient, the method comprising measuring viscoelasticity of liver tissue from the patient, wherein increased viscoelasticity of the liver tissue of the patient compared to reference value ranges for viscoelasticity of liver tissue from a control subject indicates that the patient is at risk of developing HCC or has a positive diagnosis for the HCC.
9. A method of generating a non-human animal model of non-alcoholic steatohepatitis with hydrodynamically induced hepatocellular carcinoma (HCC), the method comprising: administering an advanced glycation end-product to the non-human animal, wherein viscoelasticity of liver tissue increases in the non-human animal.
10. The method of aspect 9, wherein the non-human animal is a mammal.
11. The method of aspect 10, wherein the mammal is a mouse.
12. The non-human animal model of non-alcoholic steatohepatitis with hydrodynamically induced hepatocellular carcinoma (HCC) produced by the method of any one of aspects 9-11.
13. A method of screening a candidate agent, the method comprising:
administering the candidate agent to the non-human animal model of aspect 12; and
measuring viscoelasticity of liver tissue in the non-human animal model.
14. The method of aspect 13, further comprising determining if the candidate agent is an AGE inhibitor or a crosslink breaker or increases expression or activity of AGER1.
15. The method of aspect 13 or 14, further comprising determining if the candidate agent has anti-cancer activity.
16. An isolated cancerous liver cell from the non-human animal model of aspect 12.
17. A method of screening a candidate agent, the method comprising:
contacting the cancerous liver cell of aspect 16 with the candidate agent; and
measuring the viscoelasticity of the ECM of the cancerous liver cell.
18. The method of aspect 17, further comprising testing if the candidate agent is an AGE inhibitor or a crosslink breaker or increases expression or activity of AGER1.
19. The method of aspect 17 or 18, further comprising testing if the candidate agent has anti-cancer activity.
20. A method of treating a subject for hepatocellular carcinoma (HCC), the method comprising administering a therapeutically effective amount of an advanced glycation end product (AGE) inhibitor, a crosslink breaker that cleaves crosslinks formed by an AGE, or a combination thereof to the subject.
21. The method of aspect 20, wherein the AGE inhibitor is pyridoxamine.
22. The method of aspect 20 or 21, wherein the crosslink breaker is alagebrium (3-phenacyl-4,5-dimethylthiazolium chloride).
23. The method of any one of aspects 20-22, wherein multiple cycles of treatment are administered to said subject for a time period sufficient to effect at least a partial tumor response.
24. The method of aspect 23, wherein the time period is at least 6 months.
25. The method of aspect 24, wherein the time period is at least 12 months.
26. The method of any one of aspects 23-25, wherein a complete tumor response is effected.
27. The method of any one of aspects 20-26, wherein treatment results in a reduction in tumor size, a reduction in the number cancerous cells, slowing or halting of tumor growth, slowing or halting of cancer cell infiltration into peripheral organs, slowing or halting of tumor metastasis, or a combination thereof.
28. The method of any one of aspects 20-27, wherein the AGE inhibitor, the crosslink breaker, or the combination thereof are administered according to a daily dosing regimen or intermittently.
29. A method of treating a subject for hepatocellular carcinoma (HCC), the method comprising administering a vector comprising a promoter operably linked to a nucleotide sequence comprising a coding sequence encoding AGER1 to the subject, wherein a therapeutically effective amount of the AGER1 is expressed in vivo in the subject.
30. The method of aspect 29, wherein the vector is administered intravenously, intra-arterially, or locally into the liver.
31. The method of aspect 29 or 30, wherein the vector is a viral vector.
32. The method of aspect 31, wherein the viral vector is an adeno-associated virus (AAV) vector.
33. The method of any one of aspects 29-32, wherein the promoter is a constitutive or inducible promoter.
34. A method of providing a subject with AGER1, the method comprising introducing a vector comprising a promoter operably linked to a coding sequence encoding the AGER1 into a liver cell, wherein the cell expresses the AGER1 in vivo in the subject in an effective amount sufficient to have anti-cancer activity.
35. The method of aspect 34, wherein the vector is introduced into the cell ex vivo or in vivo.
36. The method of aspect 34 or 35, wherein the vector is a viral vector.
37. The method of aspect 36, wherein the viral vector is an adeno-associated virus (AAV) vector.
38. The method of any one of aspects 34-37, wherein the promoter is a constitutive or inducible promoter.
39. A composition for use in a method of treating hepatocellular carcinoma (HCC), the composition comprising an advanced glycation end product (AGE) inhibitor, a crosslink breaker that cleaves crosslinks formed by an AGE, or a combination thereof.
40. The composition of aspect 39, wherein the AGE inhibitor is pyridoxamine.
41. The composition of aspect 39 or 40, wherein the crosslink breaker is alagebrium (3-phenacyl-4,5-dimethylthiazolium chloride).
42. The composition of any one of aspects 39-41, further comprising a pharmaceutically acceptable excipient.
43. A composition for use in a method of treating hepatocellular carcinoma (HCC), the composition comprising AGER1 or a vector comprising a promoter operably linked to a coding sequence encoding AGER1.
44. The composition of aspect 43, wherein the viral vector is an adeno-associated virus (AAV) vector.
45. The composition of aspect 43 or 44, further comprising a pharmaceutically acceptable excipient.

It will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### Extracellular Matrix Viscoelasticity Drives Hepatocellular Carcinoma Progression

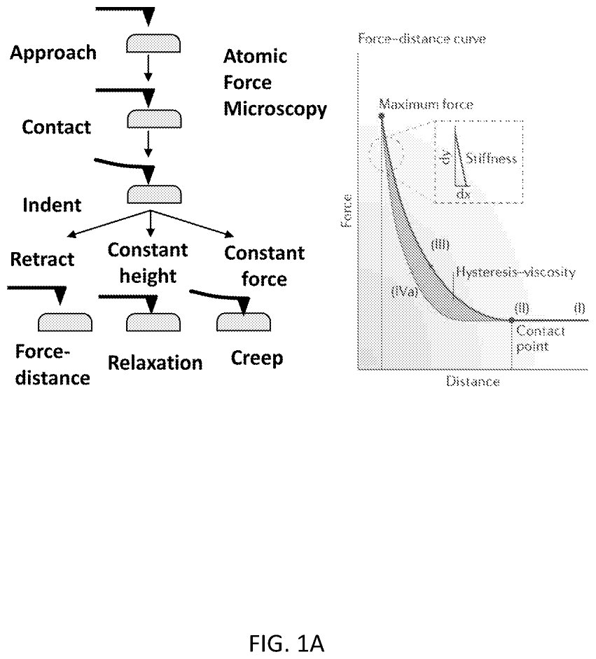

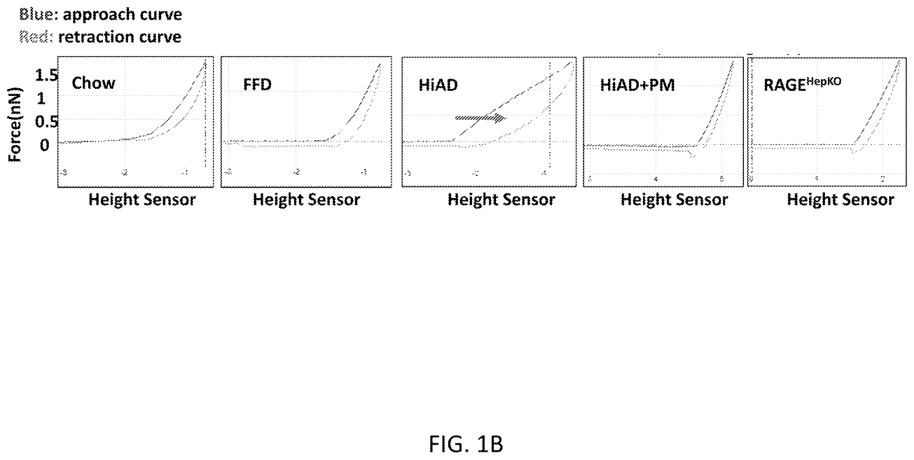

As shown in FIGS. 1A-1B, extracellular matrix (ECM) viscoelasticity increases in an AGE and hepatocyte RAGE-dependent manner. Mechanical responses to indentation were assessed by an atomic force microscope (AFM) probe. A force-distance curve is generated when the probe indents the sample until a defined force is reached and the cantilever is retracted. The hysteresis between the approach and retraction curves indicates sample viscosity (FIG. 1A). Force curves using livers from HiAD-fed mice exhibited a larger hysteresis area compared to chow or FFD-fed mice. The hysteresis is also reduced in PM-treated (60 mg/kg daily, i.p.) or in RAGE HepKO mice (FIG. 1B, N=2-3/group).

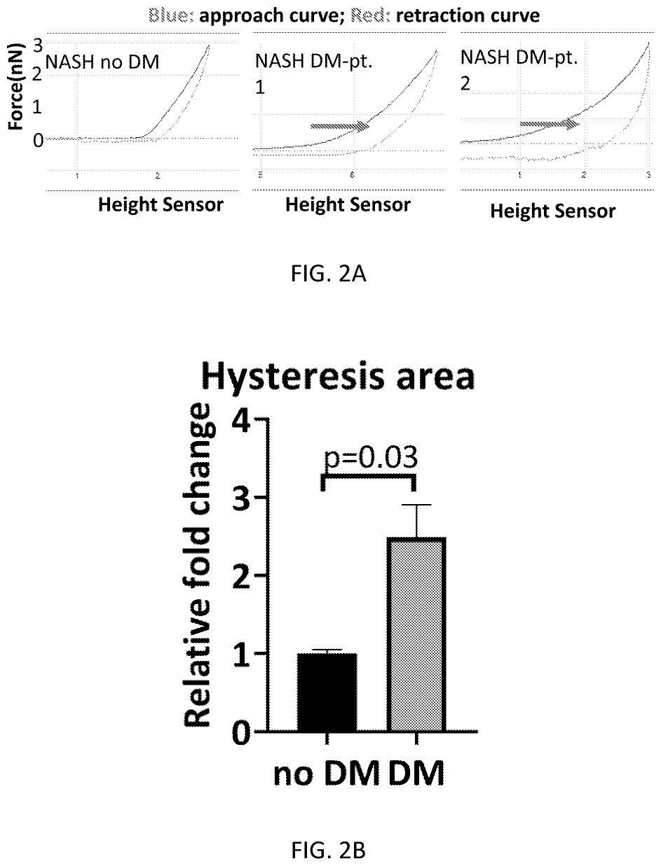

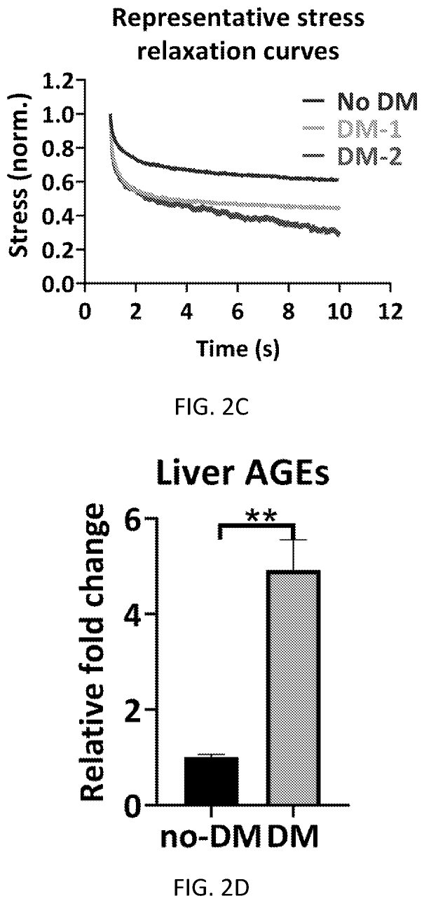

As shown in FIGS. 2A-2D, atomic force microscopy on non-cirrhotic liver samples from NASH patients showed higher viscoelasticity in patients with type 2 diabetes mellitus (T2DM). Force curves showed a larger hysteresis area (the area between approach and retraction force curves) in patients with T2DM (FIG. 2A). A Trapz function in MATLAB was used to measure the hysteresis area in (FIG. 2A), N=10 areas in each group, each sample (FIG. 2B, mean±SEM, p=0.03, ANOVA, Tukey's post hoc). Representative stress relaxation response curves showed faster stress relaxation in NASH patients with T2DM (FIG. 2C). In this test, a constant strain (3 nN) was applied and the stress in response to strain was measured for 10 s. Liver AGEs increased in patients with T2DM (n=3 in each group (FIG. 2D).

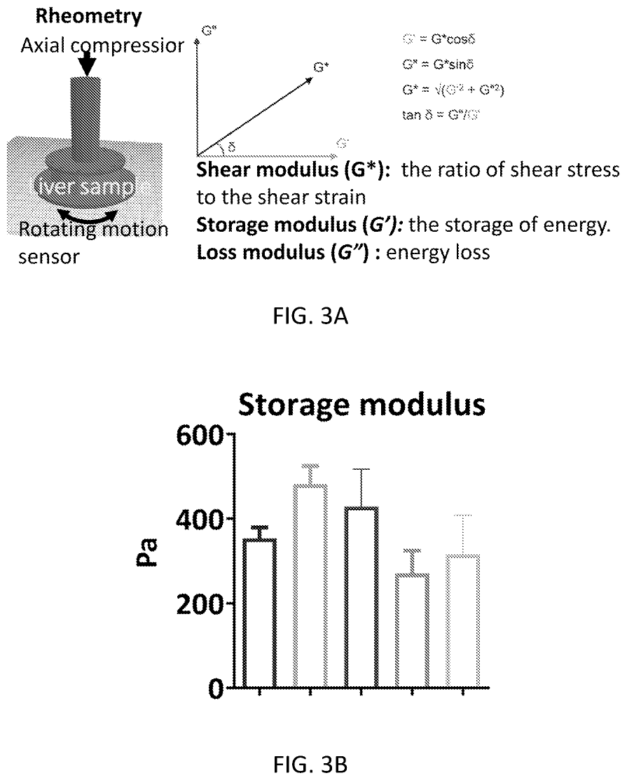

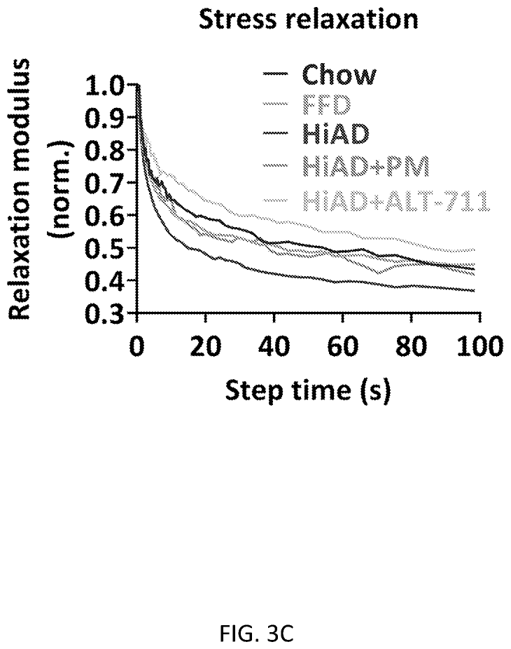

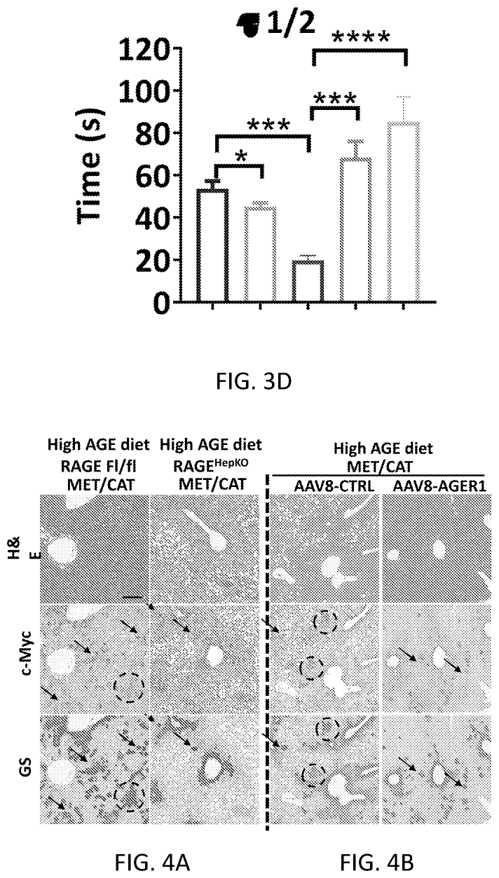

As shown in FIGS. 3A-3D, rheometry results showed mice on HiAD exhibited faster relaxation times (high viscoelasticity) that was reversed by PM, and alagebrium (ALT-711). Stiffness did not change. The plate was lowered to touch the sample and 100˜300 Pa of initial force was applied. Dynamic time sweep test (2% constant strain, oscillation frequency 1 radian/s, measurements taken for 600 s) was done first to collect the shear storage modulus (stiffness, G′) and loss modulus (G″) (FIG. 3A). The storage modulus (stiffness) did not show significant change (FIG. 3B). A stress relaxation test was performed for 600 s with an initial 10% strain. Livers from HiAD-fed mice exhibited faster stress relaxation compared to chow, FFD-fed mice or those treated with the AGE inhibitor, PM, or ALT-711 (crosslink breaker). Stress was normalized by the initial stress (FIG. 3C). Quantification of timescale at which the stress is relaxed to half its original value (τ_{1/2}), from the tests in FIG. 3C (FIG. 3D). (N=2-4, Data are represented as mean±SEM; one way ANOVA followed by post-hoc Tukey test *p<0.05, **p<0.01, ***p<0.001).

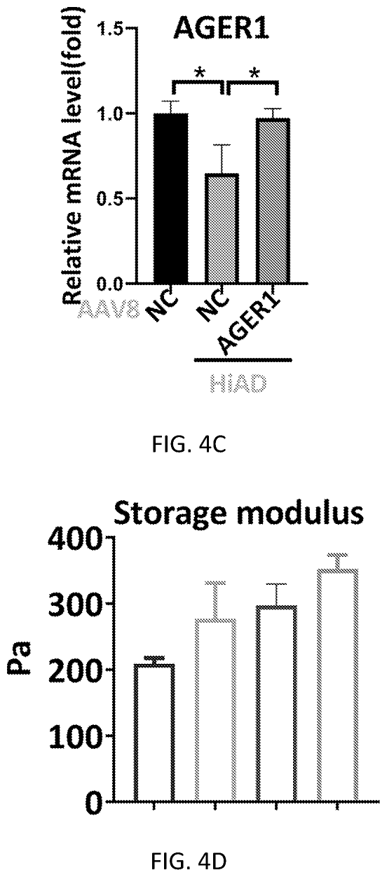

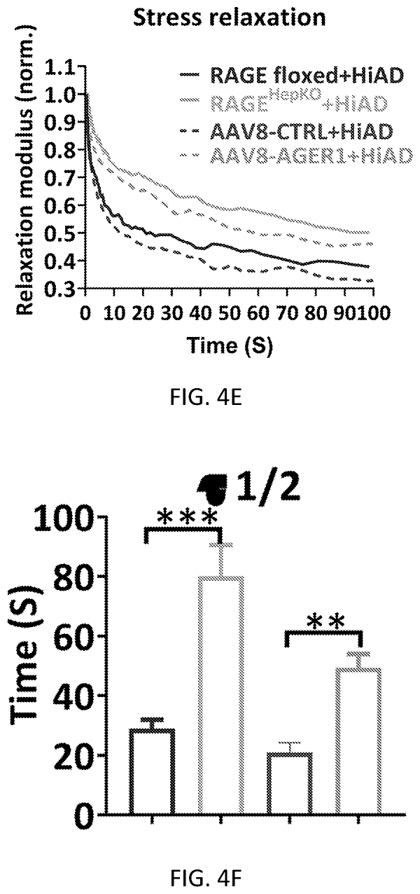

As shown in FIGS. 4A-4F, RAGE^{HepKO} and AGER1 reconstituted mice exhibited lower numbers of GS/c-Myc positive transformed cells. RAGE fl/fl or RAGE^{HepKO} mice were fed HiAD for 7 weeks, HD-injected, and sacrificed 4 weeks later (FIG. 4A, bar=100 μm). AGER1 was reconstituted by injecting AAV8-TBG-AGER1 mice via their tail vein 2 weeks prior to the HD injection. As a control AAV8-TBG-GFP were used (FIG. 4B). GS/c-Myc positive cells decreased in RAGE^{HepKO} mice and AAV8-AGER1 mice (FIGS. 4A, 4B). RT-qPCR showed increased AGER1 levels (FIG. 4C). Rheometry data demonstrate that the storage modulus (stiffness) did not change (FIG. 4D). Livers from RAGE^{HepKO} and AAV8-TBG-AGER1 mice exhibited faster stress relaxation compared to control mice. Stress was normalized by the initial stress (FIG. 4E). FIG. 4F shows the quantification of the timescale at which the stress is relaxed to half its original value (τ½), from stress relaxation tests in FIG. 4C. (N=4-6, Data are represented as mean±SEM; one way ANOVA followed by post-hoc Tukey test *p<0.05, **p<0.01, ***p<0.001).

1. A method of diagnosing and treating hepatocellular carcinoma (HCC) in a patient, the method comprising:
measuring viscoelasticity of liver tissue from the patient, wherein increased viscoelasticity of the liver tissue of the patient compared to reference value ranges for viscoelasticity of liver tissue from a control subject indicates that the patient is at risk of developing the HCC or has a positive diagnosis for the HCC; and
treating the patient for the HCC, if the patient has a positive diagnosis for the HCC based on the measured viscoelasticity of the liver tissue.

2. The method of claim 1, wherein the patient has non-alcoholic steatohepatitis or type 2 diabetes mellitus, or a combination thereof.

3-4. (canceled)

5. The method of claim 1, wherein said treating the patient for HCC comprises surgical resection of an HCC tumor, liver transplantation, radiofrequency ablation, cryoablation, radiation therapy, chemotherapy, immunotherapy, or biologic therapy, or a combination thereof.

6-8. (canceled)

9. A method of generating a non-human animal model of non-alcoholic steatohepatitis with hydrodynamically induced hepatocellular carcinoma (HCC), the method comprising: administering an advanced glycation end-product to the non-human animal, wherein viscoelasticity of liver tissue increases in the non-human animal.

10. The method of claim 9, wherein the non-human animal is a mammal.

11. The method of claim 10, wherein the mammal is a mouse.

12. The non-human animal model of non-alcoholic steatohepatitis with hydrodynamically induced hepatocellular carcinoma (HCC) produced by the method of claim 9.

13. A method of screening a candidate agent, the method comprising:
administering the candidate agent to the non-human animal model of claim 12; and
measuring viscoelasticity of liver tissue in the non-human animal model.

14. The method of claim 13, further comprising determining if the candidate agent is an AGE inhibitor or a crosslink breaker, or increases expression or activity of AGER1, or has anti-cancer activity.

15. (canceled)

16. An isolated cancerous liver cell from the non-human animal model of claim 12.

17. A method of screening a candidate agent, the method comprising:
contacting the cancerous liver cell of claim 16 with the candidate agent; and
measuring the viscoelasticity of the extracellular matrix (ECM) of the cancerous liver cell.

18. The method of claim 17, further comprising testing if the candidate agent is an AGE inhibitor or a crosslink breaker, or increases expression or activity of AGER1, or has anti-cancer activity.

19. (canceled)

20. A method of treating a subject for hepatocellular carcinoma (HCC), the method comprising administering a therapeutically effective amount of an advanced glycation end product (AGE) inhibitor, a crosslink breaker that cleaves crosslinks formed by an AGE, or a combination thereof to the subject.

21. The method of claim 20, wherein the AGE inhibitor is pyridoxamine.

22. The method of claim 20, wherein the crosslink breaker is alagebrium (3-phenacyl-4,5-dimethylthiazolium chloride).

23. The method of claim 20, wherein multiple cycles of treatment are administered to said subject for a time period sufficient to effect at least a partial tumor response.

24-28. (canceled)

29. A method of treating a subject for hepatocellular carcinoma (HCC), the method comprising administering a vector comprising a promoter operably linked to a nucleotide sequence comprising a coding sequence encoding AGER1 to the subject, wherein a therapeutically effective amount of the AGER1 is expressed in vivo in the subject.

30. The method of claim 29, wherein the vector is administered intravenously, intra-arterially, or locally into the liver.

31. The method of claim 29, wherein the vector is a viral vector.

32. The method of claim 31, wherein the viral vector is an adeno-associated virus (AAV) vector.

33-45. (canceled)


SEQUENCE LISTING (1 sequence)

SEQ ID NO: 1 - protein, 439 aa, Homo sapiens
MEPSTAARAWALFWLLLPLLGAVCASGPRTLVLLDNLNVRETHSLFFRSLKDRGFELTFKTADDPSLSLIKYGEFLYDNLIIFSPSVEDFGGNINVETISAFIDGGGSVLVAASSDIGDPLRELGSECGIEFDEEKTAVIDHHNYDISDLGQHTLIVADTENLLKAPTIVGKSSLNPILFRGVGMVADPDNPLVLDILTGSSTSYSFFPDKPITQYPHAVGKNTLLIAGLQARNNARVIFSGSLDFFSDSFFNSAVQKAAPGSQRYSQTGNYELAVALSRWVFKEEGVLRVGPVSHHRVGETAPPNAYTVTDLVEYSIVIQQLSNGKWVPFDGDDIQLEFVRIDPFVRTFLKKKGGKYSVQFKLPDVYGVFQFKVDYNRLGYTHLYSSTQVSVRPLQHTQYERFIPSAYPYYASAFSMMLGLFIFSIVFLHMKEKEKSD